SEQUENCE LISTING (248 sequences)

SEQ ID NO: 1 - protein, 5 aa, Artificial Sequence
  Human antibody library
RYHMM

SEQ ID NO: 2 - protein, 7 aa, Artificial Sequence
  Human antibody library
GFTFSRY

SEQ ID NO: 3 - protein, 17 aa, Artificial Sequence
  Human antibody library
SIYPSGGYTYYADSVKG

SEQ ID NO: 4 - protein, 6 aa, Artificial Sequence
  Human antibody library
YPSGGY

SEQ ID NO: 5 - protein, 8 aa, Artificial Sequence
  Human antibody library
EYFSGPDY

SEQ ID NO: 6 - protein, 117 aa, Artificial Sequence
  Human antibody library
EVQLLESGGGLVQPGGSLRLSCAASGFTFSRYHMMWVRQAPGKGLEWVSSIYPSGGYTYYADSVKGRFTISRDNSKNTLYLQMNSLRAEDTAVYYCAREYFSGPDYWGQGTLVTVSS

SEQ ID NO: 7 - protein, 11 aa, Artificial Sequence
  Human antibody library
RASQKISRWLA

SEQ ID NO: 8 - protein, 7 aa, Artificial Sequence
  Human antibody library
RASDLES

SEQ ID NO: 9 - protein, 9 aa, Artificial Sequence
  Human antibody library
QQYNTYPYT

SEQ ID NO: 10 - protein, 108 aa, Artificial Sequence
  Human antibody library
DIQMTQSPSTLSASVGDRVTITCRASQKISRWLAWYQQTPGKAPKLLIYRASDLESGVPSRFSGSGSGTEFTLTISSLQPDDFATYYCQQYNTYPYTFGQGTKLEIKR

SEQ ID NO: 11 - protein, 5 aa, Artificial Sequence
  Human antibody library
WYSML

SEQ ID NO: 12 - protein, 7 aa, Artificial Sequence
  Human antibody library
GFTFSWY

SEQ ID NO: 13 - protein, 17 aa, Artificial Sequence
  Human antibody library
RIYPSGGFTYYADSVKG

SEQ ID NO: 14 - protein, 6 aa, Artificial Sequence
  Human antibody library
YPSGGF

SEQ ID NO: 15 - protein, 17 aa, Artificial Sequence
  Human antibody library
VIDIVGATGRLQHGMDV

SEQ ID NO: 16 - protein, 126 aa, Artificial Sequence
  Human antibody library
EVQLLESGGGLVQPGGSLRLSCAASGFTFSWYSMLWVRQAPGKGLEWVSRIYPSGGFTYYADSVKGRFTISRDNSKNTLYLQMNSLRAEDTAVYYCARVIDIVGATGRLQHGMDVWGQGTTVTVSS

SEQ ID NO: 17 - protein, 11 aa, Artificial Sequence
  Human antibody library
GGNNIGSKSVN

SEQ ID NO: 18 - protein, 7 aa, Artificial Sequence
  Human antibody library
DDSDRPS

SEQ ID NO: 19 - protein, 11 aa, Artificial Sequence
  Human antibody library
QVWDSVSDHYV

SEQ ID NO: 20 - protein, 111 aa, Artificial Sequence
  Human antibody library
QYELTQPPSVSVAPGQTAKITCGGNNIGSKSVNWYQQKTGQAPVLVVHDDSDRPSGIPERLSGSNSGNTATLTISRAEAGDEADYYCQVWDSVSDHYVFGTGTKVTVLGQP

SEQ ID NO: 21 - protein, 5 aa, Artificial Sequence
  Human antibody library
YYWMS

SEQ ID NO: 22 - protein, 7 aa, Artificial Sequence
  Human antibody library
GFTFSYY

SEQ ID NO: 23 - protein, 17 aa, Artificial Sequence
  Human antibody library
YISPSGGMTKYADSVKG

SEQ ID NO: 24 - protein, 6 aa, Artificial Sequence
  Human antibody library
SPSGGM

SEQ ID NO: 25 - protein, 17 aa, Artificial Sequence
  Human antibody library
ASYDYVWGIYHYDVFDI

SEQ ID NO: 26 - protein, 126 aa, Artificial Sequence
  Human antibody library
EVQLLESGGGLVQPGGSLRLSCAASGFTFSYYWMSWVRQAPGKGLEWVSYISPSGGMTKYADSVKGRFTISRDNSKNTLYLQMNSLRAEDTAVYYCASASYDYVWGIYHYDVFDIWGQGTMVTVSS

SEQ ID NO: 27 - protein, 11 aa, Artificial Sequence
  Human antibody library
RASQGISSWLA

SEQ ID NO: 28 - protein, 7 aa, Artificial Sequence
  Human antibody library
AASSLQS

SEQ ID NO: 29 - protein, 9 aa, Artificial Sequence
  Human antibody library
QQANSFPLT

SEQ ID NO: 30 - protein, 108 aa, Artificial Sequence
  Human antibody library
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKR

SEQ ID NO: 31 - protein, 5 aa, Artificial Sequence
  Human antibody library
DYNMA

SEQ ID NO: 32 - protein, 7 aa, Artificial Sequence
  Human antibody library
GFTFSDY

SEQ ID NO: 33 - protein, 17 aa, Artificial Sequence
  Human antibody library
SIGPSGGNTHYADSVKG

SEQ ID NO: 34 - protein, 6 aa, Artificial Sequence
  Human antibody library
GPSGGN

SEQ ID NO: 35 - protein, 12 aa, Artificial Sequence
  Human antibody library
ERVEYYYYGMDV

SEQ ID NO: 36 - protein, 121 aa, Artificial Sequence
  Human antibody library
EVQLLESGGGLVQPGGSLRLSCAASGFTFSDYNMAWVRQAPGKGLEWVSSIGPSGGNTHYADSVKGRFTISRDNSKNTLYLQMNSLRAEDMAVYYCARERVEYYYYGMDVWGKGTTVTVSS

SEQ ID NO: 37 - protein, 11 aa, Artificial Sequence
  Human antibody library
GGDNIESLSVH

SEQ ID NO: 38 - protein, 7 aa, Artificial Sequence
  Human antibody library
DDADRPS

SEQ ID NO: 39 - protein, 11 aa, Artificial Sequence
  Human antibody library
QVWDYSSDVVV

SEQ ID NO: 40 - protein, 111 aa, Artificial Sequence
  Human antibody library
QSALTQAPSVSVAPGQTARITCGGDNIESLSVHWYQQKPGQAPVLVVYDDADRPSGTPERFSGSNSGNTATLTINRAEAGDEADYYCQVWDYSSDVVVFGGGTTLTVLGQP

SEQ ID NO: 41 - protein, 11 aa, Artificial Sequence
  Human antibody library
RASQSISSWLA

SEQ ID NO: 42 - protein, 7 aa, Artificial Sequence
  Human antibody library
KASSLDS

SEQ ID NO: 43 - protein, 9 aa, Artificial Sequence
  Human antibody library
QQYDSYPYT

SEQ ID NO: 44 - protein, 108 aa, Artificial Sequence
  Human antibody library
DIQMTQSPSTLSASVGDRVTITCRASQSISSWLAWYQQKPGKAPNLLIYKASSLDSGVPPRFSGSGSGTEFTLTISSLQPDDFATYYCQQYDSYPYTFGQGTKLEIKR

SEQ ID NO: 45 - protein, 11 aa, Artificial Sequence
  Human antibody library
RASQSISSWLA

SEQ ID NO: 46 - protein, 7 aa, Artificial Sequence
  Human antibody library
KASSLES

SEQ ID NO: 47 - protein, 9 aa, Artificial Sequence
  Human antibody library
QQYDNYSYT

SEQ ID NO: 48 - protein, 108 aa, Artificial Sequence
  Human antibody library
DIQMTQSPSTLSASVGDRVTITCRASQSISSWLAWYQQKPGKAPKLLISKASSLESGVPSRFSGSGSGTEFTLTISSLQPDDFATYYCQQYDNYSYTFGQGTKLEIKR

SEQ ID NO: 49 - protein, 11 aa, Artificial Sequence
  Human antibody library
RASQSISSWLA

SEQ ID NO: 50 - protein, 7 aa, Artificial Sequence
  Human antibody library
KASSLQT

SEQ ID NO: 51 - protein, 9 aa, Artificial Sequence
  Human antibody library
QQYNSYPYT

SEQ ID NO: 52 - protein, 108 aa, Artificial Sequence
  Human antibody library
DIQMTQSPSALSASVGDRVTITCRASQSISSWLAWYQQKPGRAPKLLIYKASSLQTGVPSRFSGSGSGTDLALTISSLQPDDFATYYCQQYNSYPYTFGQGTRLELKR

SEQ ID NO: 53 - protein, 11 aa, Artificial Sequence
  Human antibody library
RASQTISSWLA

SEQ ID NO: 54 - protein, 7 aa, Artificial Sequence
  Human antibody library
KASSLES

SEQ ID NO: 55 - protein, 9 aa, Artificial Sequence
  Human antibody library
QQYDGYPYT

SEQ ID NO: 56 - protein, 108 aa, Artificial Sequence
  Human antibody library
DIQMTQSPSTLSASVGDRVTITCRASQTISSWLAWYQQKPGKAPKLLIYKASSLESGVPSRFSGSGSGTEFTLTISSLQPDDFATYYCQQYDGYPYTFGQGTRLEIKR

SEQ ID NO: 57 - protein, 11 aa, Artificial Sequence
  Human antibody library
RASQSIDSWLA

SEQ ID NO: 58 - protein, 7 aa, Artificial Sequence
  Human antibody library
KASNLEG

SEQ ID NO: 59 - protein, 9 aa, Artificial Sequence
  Human antibody library
QQYDSYPYT

SEQ ID NO: 60 - protein, 108 aa, Artificial Sequence
  Human antibody library
DIQMTQSPSTLSASVGDRVTIACRASQSIDSWLAWYQQKPGKAPKLLIYKASNLEGGVPSRFSGSGSGTEFTLTISSLQPDDFATYYCQQYDSYPYTFGQGTELGIKR

SEQ ID NO: 61 - protein, 11 aa, Artificial Sequence
  Human antibody library
RASEIVSSWLA

SEQ ID NO: 62 - protein, 7 aa, Artificial Sequence
  Human antibody library
KASTLEA

SEQ ID NO: 63 - protein, 9 aa, Artificial Sequence
  Human antibody library
QQYNDYPYT

SEQ ID NO: 64 - protein, 108 aa, Artificial Sequence
  Human antibody library
DIQMTQSPSTLSASVGDKVTITCRASEIVSSWLAWYQQTPGRAPKLLISKASTLEAGVPSRFSGSGSGTDFTLTIGSLQPDDFGTYYCQQYNDYPYTFGQGTRLDIRR

SEQ ID NO: 65 - protein, 11 aa, Artificial Sequence
  Human antibody library
RASQDIDRWLA

SEQ ID NO: 66 - protein, 7 aa, Artificial Sequence
  Human antibody library
RVSDLEN

SEQ ID NO: 67 - protein, 9 aa, Artificial Sequence
  Human antibody library
QHYDRYPYT

SEQ ID NO: 68 - protein, 108 aa, Artificial Sequence
  Human antibody library
DIQMTQSPSTLSASVGDRVTITCRASQDIDRWLAWYQQKPGKAPKLLIYRVSDLENGVPSRFSGSGSGTEFTLTISSLQPDDSATYYCQHYDRYPYTFGQGTKLEIKR

SEQ ID NO: 69 - protein, 11 aa, Artificial Sequence
  Human antibody library
RASQSISRWLA

SEQ ID NO: 70 - protein, 7 aa, Artificial Sequence
  Human antibody library
KASSLET

SEQ ID NO: 71 - protein, 9 aa, Artificial Sequence
  Human antibody library
QHYDSYPYT

SEQ ID NO: 72 - protein, 108 aa, Artificial Sequence
  Human antibody library
DIQMTQSPSTLSASVGDRVTITCRASQSISRWLAWYQQKPGRAPRVLIYKASSLETGVPSRFSGSGSGTEFTLTINSLQPDDFATYYCQHYDSYPYTFGQGTKLEIKR

SEQ ID NO: 73 - protein, 11 aa, Artificial Sequence
  Human antibody library
RASQSISRWLA

SEQ ID NO: 74 - protein, 7 aa, Artificial Sequence
  Human antibody library
KASTLGI

SEQ ID NO: 75 - protein, 9 aa, Artificial Sequence
  Human antibody library
QQYDTYPYT

SEQ ID NO: 76 - protein, 108 aa, Artificial Sequence
  Human antibody library
DIQMTQSPSTLSASVGDRVTISCRASQSISRWLAWYQQKPGKAPKLLIYKASTLGIEVPSRFSGSGSGTEFTLTVSSLQPDDFATYYCQQYDTYPYTFGQGTKLEIKR

SEQ ID NO: 77 - protein, 14 aa, Artificial Sequence
  Human antibody library
TGSSSDIGDYNYVS

SEQ ID NO: 78 - protein, 7 aa, Artificial Sequence
  Human antibody library
EVSNRPS

SEQ ID NO: 79 - protein, 11 aa, Artificial Sequence
  Human antibody library
QVWDSSSDHYV

SEQ ID NO: 80 - protein, 114 aa, Artificial Sequence
  Human antibody library
  misc_feature (9)..(9): Xaa can be any naturally occurring amino acid
QSALTQPAXVSGSPGQSITISCTGSSSDIGDYNYVSWYQQYPGKAPKLMIFEVSNRPSGVSSRFSGSNSGNTATLTISRVEAGDEADYYCQVWDSSSDHYVFGTGTKVNVLGQP

SEQ ID NO: 81 - protein, 11 aa, Artificial Sequence
  Human antibody library
GGHNIGTENVH

SEQ ID NO: 82 - protein, 7 aa, Artificial Sequence
  Human antibody library
DDNNRPS

SEQ ID NO: 83 - protein, 11 aa, Artificial Sequence
  Human antibody library
QVWDISTDQYV

SEQ ID NO: 84 - protein, 111 aa, Artificial Sequence
  Human antibody library
QSALTQPPSVSVAPGQTARITCGGHNIGTENVHWYQQKPGQAPVLVVFDDNNRPSGIPERFSGSNSGNTATLTINRVEAGDEADFYCQVWDISTDQYVFGPGTRVTVLGQP

SEQ ID NO: 85 - protein, 11 aa, Artificial Sequence
  Human antibody library
GGNNIGDKSVH

SEQ ID NO: 86 - protein, 7 aa, Artificial Sequence
  Human antibody library
EDKKRPA

SEQ ID NO: 87 - protein, 11 aa, Artificial Sequence
  Human antibody library
QVWDFTTDQFV

SEQ ID NO: 88 - protein, 111 aa, Artificial Sequence
  Human antibody library
QSALTQPPSVSLAPGQTARISCGGNNIGDKSVHWYRQKPGQAPAVVVYEDKKRPAGIPERLSGSNSGDTATLTIDRVEAGDEADYYCQVWDFTTDQFVFGSGTKVTVLSQP

SEQ ID NO: 89 - protein, 11 aa, Artificial Sequence
  Human antibody library
GGNNLGDRPVH

SEQ ID NO: 90 - protein, 7 aa, Artificial Sequence
  Human antibody library
EDKKRPG

SEQ ID NO: 91 - protein, 11 aa, Artificial Sequence
  Human antibody library
QVWDFITDQFV

SEQ ID NO: 92 - protein, 111 aa, Artificial Sequence
  Human antibody library
QSALTQPPSVSASPGQTARISCGGNNLGDRPVHWYRQKPGQAPVVVVYEDKKRPGGIPERLSGSTSDDTATLTIDRVEAGDEADYYCQVWDFITDQFVFGSGTTLTVLSQP

SEQ ID NO: 93 - protein, 11 aa, Artificial Sequence
  Human antibody library
GGNNIGGKSVH

SEQ ID NO: 94 - protein, 7 aa, Artificial Sequence
  Human antibody library
YDADRPS

SEQ ID NO: 95 - protein, 11 aa, Artificial Sequence
  Human antibody library
QVWDGTSDHYV

SEQ ID NO: 96 - protein, 111 aa, Artificial Sequence
  Human antibody library
QSELTQPPSVSVAPGQTATLPCGGNNIGGKSVHWYQQKPGQAPGLVIYYDADRPSGIPERFSGSNSGNTATLTIDRVEVGDEADYYCQVWDGTSDHYVFGTGTKLTVLGQP

SEQ ID NO: 97 - protein, 11 aa, Artificial Sequence
  Human antibody library
GGNNIGGKSVN

SEQ ID NO: 98 - protein, 7 aa, Artificial Sequence
  Human antibody library
YDNARPS

SEQ ID NO: 99 - protein, 11 aa, Artificial Sequence
  Human antibody library
QVWDSSSDHYV

SEQ ID NO: 100 - protein, 111 aa, Artificial Sequence
  Human antibody library
QSVLTQSPSVSVAPGETARVTCGGNNIGGKSVNWYQQKSGQAPVLVIYYDNARPSGIPERISGSNSGNTATLTISSVEAGDEADYYCQVWDSSSDHYVFGTGTKVTVLGQP

SEQ ID NO: 101 - protein, 11 aa, Artificial Sequence
  Human antibody library
GGNNIGYKSVH

SEQ ID NO: 102 - protein, 7 aa, Artificial Sequence
  Human antibody library
DDSDRPS

SEQ ID NO: 103 - protein, 11 aa, Artificial Sequence
  Human antibody library
QVWDAPTDHVV

SEQ ID NO: 104 - protein, 111 aa, Artificial Sequence
  Human antibody library
QYELTQPPSVSVAPGKAARIPCGGNNIGYKSVHWYQQKPGQAPVLVVYDDSDRPSGIPERFSGSNSGNTATLTISRVEAGDEADYYCQVWDAPTDHVVFGGGTKLSVLGQP

SEQ ID NO: 105 - protein, 11 aa, Artificial Sequence
  Human antibody library
GGNNIGKESVH

SEQ ID NO: 106 - protein, 7 aa, Artificial Sequence
  Human antibody library
YDSERPS

SEQ ID NO: 107 - protein, 11 aa, Artificial Sequence
  Human antibody library
QVWDSRNDHVV

SEQ ID NO: 108 - protein, 111 aa, Artificial Sequence
  Human antibody library
QSALTQPPSVSVAPGKTARITCGGNNIGKESVHWYQKNPGQAPVLVIYYDSERPSGIPERFSGSNSGNTATLTISRVEAGDEADYFCQVWDSRNDHVVFGGGTKLTVLSQP

SEQ ID NO: 109 - protein, 11 aa, Artificial Sequence
  Human antibody library
SGDKLGNKYTS

SEQ ID NO: 110 - protein, 7 aa, Artificial Sequence
  Human antibody library
EDNKRPS

SEQ ID NO: 111 - protein, 11 aa, Artificial Sequence
  Human antibody library
QVWDFITDQFV

SEQ ID NO: 112 - protein, 111 aa, Artificial Sequence
  Human antibody library
QYELTQPPSVSVSPGQTASITCSGDKLGNKYTSWYQQKPGQSPILVIYEDNKRPSGIPERFSGSNSGNTATLTIDRVEAGDEADYYCQVWDFITDQFVFGSGTTLTVLSQP

SEQ ID NO: 113 - protein, 11 aa, Artificial Sequence
  Human antibody library
SGDKLGDKYVA

SEQ ID NO: 114 - protein, 7 aa, Artificial Sequence
  Human antibody library
EDNKRPS

SEQ ID NO: 115 - protein, 11 aa, Artificial Sequence
  Human antibody library
QAWDRSTDHYV

SEQ ID NO: 116 - protein, 111 aa, Artificial Sequence
  Human antibody library
QSVLTQPPSVSVSPGQTATIICSGDKLGDKYVAWYQQKPGQSPVLVVYEDNKRPSGIPERISGSNSGNTATLTISGTQAMDDADYYCQAWDRSTDHYVFGTGTKVTVLGQP

SEQ ID NO: 117 - protein, 11 aa, Artificial Sequence
  Human antibody library
GGDKIGSQSVH

SEQ ID NO: 118 - protein, 7 aa, Artificial Sequence
  Human antibody library
YDDVRPS

SEQ ID NO: 119 - protein, 11 aa, Artificial Sequence
  Human antibody library
QVWDFSTEHVV

SEQ ID NO: 120 - protein, 111 aa, Artificial Sequence
  Human antibody library
QSELTQPPSVSVAPGETATITCGGDKIGSQSVHWYQQRPGQAPVQVVFYDDVRPSGIPERISGSKSGNSATLTISRVEGGDEADYYCQVWDFSTEHVVFGGGTSLTVLSQP

SEQ ID NO: 121 - protein, 11 aa, Artificial Sequence
  Human antibody library
GGDKIGSKSVH

SEQ ID NO: 122 - protein, 7 aa, Artificial Sequence
  Human antibody library
YDDVRPS

SEQ ID NO: 123 - protein, 11 aa, Artificial Sequence
  Human antibody library
QVWDFSSEHVV

SEQ ID NO: 124 - protein, 111 aa, Artificial Sequence
  Human antibody library
QSVLTQPPSVSVAPGKTARITCGGDKIGSKSVHWYQQKPGQAPVQVIFYDDVRPSGIPERISGSKSGNSATLTISRVEGGDEADYYCQVWDFSSEHVVFGGGTSLTVLSQP

SEQ ID NO: 125 - protein, 11 aa, Artificial Sequence
  Human antibody library
SGDNLGDTYTS

SEQ ID NO: 126 - protein, 7 aa, Artificial Sequence
  Human antibody library
QDNKRAS

SEQ ID NO: 127 - protein, 11 aa, Artificial Sequence
  Human antibody library
QVWESSSDQFV

SEQ ID NO: 128 - protein, 111 aa, Artificial Sequence
  Human antibody library
QYELTQPPSVSVSPGQTASITCSGDNLGDTYTSWYQQKPGQSPVLVIYQDNKRASGIPERFSGFNSGNTATLTISGTQAVDEADYYCQVWESSSDQFVFGTGTKVTVLGQP

SEQ ID NO: 129 - protein, 11 aa, Artificial Sequence
  Human antibody library
GGDNIGSKSVH

SEQ ID NO: 130 - protein, 7 aa, Artificial Sequence
  Human antibody library
DNRDRPS

SEQ ID NO: 131 - protein, 11 aa, Artificial Sequence
  Human antibody library
QVWDPFTDHYV

SEQ ID NO: 132 - protein, 111 aa, Artificial Sequence
  Human antibody library
QSELTQPPSVSVAPGQTARISCGGDNIGSKSVHWYQQRPGQAPVLVVYDNRDRPSGIPDRFSGSISENTATLTISRVEGGDEADYYCQVWDPFTDHYVFGTGTKVTVLGQP

SEQ ID NO: 133 - protein, 11 aa, Artificial Sequence
  Human antibody library
FGYDLWDKDIS

SEQ ID NO: 134 - protein, 7 aa, Artificial Sequence
  Human antibody library
QDTKRPS

SEQ ID NO: 135 - protein, 11 aa, Artificial Sequence
  Human antibody library
QVWDASSDHYV

SEQ ID NO: 136 - protein, 111 aa, Artificial Sequence
  Human antibody library
QYELTQPPSVSVSPGQTATITCFGYDLWDKDISWYQHKAGQSPLLIMYQDTKRPSGIPERFSASNPGNTATLTINGIQAMDEADYYCQVWDASSDHYVFGTGTQVTVLGQP

SEQ ID NO: 137 - protein, 11 aa, Artificial Sequence
  Human antibody library
ERRDIGSENVH

SEQ ID NO: 138 - protein, 7 aa, Artificial Sequence
  Human antibody library
DDDKRPS

SEQ ID NO: 139 - protein, 11 aa, Artificial Sequence
  Human antibody library
QVWDSSSDHYV

SEQ ID NO: 140 - protein, 111 aa, Artificial Sequence
  Human antibody library
QYELTQPPSVSVAPGQTATIPCERRDIGSENVHWYQHKPGQAPALVVYDDDKRPSGIPARFSGSNSGDMATLTIARVEAGDEADYYCQVWDSSSDHYVFGTGTKVTVLGQP

SEQ ID NO: 141 - protein, 11 aa, Artificial Sequence
  Human antibody library
LGKDVGTKAVQ

SEQ ID NO: 142 - protein, 7 aa, Artificial Sequence
  Human antibody library
YHSDRPS

SEQ ID NO: 143 - protein, 11 aa, Artificial Sequence
  Human antibody library
QVWDSTSDHYV

SEQ ID NO: 144 - protein, 111 aa, Artificial Sequence
  Human antibody library
QSALTQPPSVSVAPGKTASISCLGKDVGTKAVQWYQQKPGQAPLLVIYYHSDRPSGIPERFSASKSGDTATLTISRVEAEDEADYYCQVWDSTSDHYVFGTGTEVTVLGQP

SEQ ID NO: 145 - protein, 11 aa, Artificial Sequence
  Human antibody library
RASQSVSSNLA

SEQ ID NO: 146 - protein, 7 aa, Artificial Sequence
  Human antibody library
GASTRAT

SEQ ID NO: 147 - protein, 11 aa, Artificial Sequence
  Human antibody library
QAYDSVADFYT

SEQ ID NO: 148 - protein, 110 aa, Artificial Sequence
  Human antibody library
DIQMTQSPATLSVSPGERATLSCRASQSVSSNLAWYQQKPGQAPRLLIYGASTRATGIPDRYNGRGSGTDFSLIISRLEPEDFASYYCQAYDSVADFYTFGQGTRVEIRR

SEQ ID NO: 149 - protein, 12 aa, Artificial Sequence
  Human antibody library
RASQSVSSNYLA

SEQ ID NO: 150 - protein, 7 aa, Artificial Sequence
  Human antibody library
GASNRVT

SEQ ID NO: 151 - protein, 10 aa, Artificial Sequence
  Human antibody library
QHYSGYPPWT

SEQ ID NO: 152 - protein, 110 aa, Artificial Sequence
  Human antibody library
DIQMTQSPATLSLSPGERATVSCRASQSVSSNYLAWYQQKPGRAPRLLMYGASNRVTGIPDRFSGSGSGTDFTLNINRLEPEDFAVYYCQHYSGYPPWTFGQGTTVEMKR

SEQ ID NO: 153 - protein, 11 aa, Artificial Sequence
  Human antibody library
RASQGISSWLA

SEQ ID NO: 154 - protein, 7 aa, Artificial Sequence
  Human antibody library
AASSLQS

SEQ ID NO: 155 - protein, 11 aa, Artificial Sequence
  Human antibody library
QQFNSYPHSFT

SEQ ID NO: 156 - protein, 110 aa, Artificial Sequence
  Human antibody library
DIQMTQSPSSVSASVGDRVTITCRASQGISSWLAWYQQKPGKAPKLLIYAASSLQSGVPSRFSGSGSGTDFTLTISSLQPEDFATYYCQQFNSYPHSFTFGPGTKVDIKR

SEQ ID NO: 157 - protein, 11 aa, Artificial Sequence
  Human antibody library
RASQGISSYLA

SEQ ID NO: 158 - protein, 7 aa, Artificial Sequence
  Human antibody library
AASTLQS

SEQ ID NO: 159 - protein, 11 aa, Artificial Sequence
  Human antibody library
QQLNIYPQRFT

SEQ ID NO: 160 - protein, 110 aa, Artificial Sequence
  Human antibody library
DIQMTQSPSFLSASVGDRVTITCRASQGISSYLAWYQQKPGKAPKLLIYAASTLQSGVPSRFSGSGSGTEFTLTISSLQPEDFATYYCQQLNIYPQRFTFGPGTKVDIKR

SEQ ID NO: 161 - protein, 11 aa, Artificial Sequence
  Human antibody library
RASQSISSYLN

SEQ ID NO: 162 - protein, 7 aa, Artificial Sequence
  Human antibody library
AASSLQS

SEQ ID NO: 163 - protein, 12 aa, Artificial Sequence
  Human antibody library
QQYGFSPPERFT

SEQ ID NO: 164 - protein, 111 aa, Artificial Sequence
  Human antibody library
DIQMTQSPSFLSASVGDRVTITCRASQSISSYLNWYQQKPGKAPKLLIYAASSLQSGVPSRFSGSGSGTDFTLTISRLESEDFAVYYCQQYGFSPPERFTFGPGTKVDIKR

SEQ ID NO: 165 - protein, 11 aa, Artificial Sequence
  Human antibody library
RASQPISGYLN

SEQ ID NO: 166 - protein, 7 aa, Artificial Sequence
  Human antibody library
AASTLQS

SEQ ID NO: 167 - protein, 11 aa, Artificial Sequence
  Human antibody library
QKYSSALEGFT

SEQ ID NO: 168 - protein, 110 aa, Artificial Sequence
  Human antibody library
DIQMTQSPSSLSASVGDTVTVTCRASQPISGYLNWYQQKPGKAPTLLIYAASTLQSGVPSRFSGSGSGTDFTLTLSSLQPEDVATYYCQKYSSALEGFTFGPGTTVDIKR

SEQ ID NO: 169 - protein, 11 aa, Artificial Sequence
  Human antibody library
RASQGISSWLA

SEQ ID NO: 170 - protein, 7 aa, Artificial Sequence
  Human antibody library
AASSLQS

SEQ ID NO: 171 - protein, 11 aa, Artificial Sequence
  Human antibody library
QKYSSALEGFT

SEQ ID NO: 172 - protein, 110 aa, Artificial Sequence
  Human antibody library
  misc_feature (8)..(10): Xaa can be any naturally occurring amino acid
DIQMTQSXXXVSASVGDRVTITCRASQGISSWLAWYQQKPGKAPKLLIYAASSLQSGVPSRFSGSGSGTDFTLTLSSLQPEDVATYYCQKYSSALEGFTFGPGTTVDIKR

SEQ ID NO: 173 - protein, 11 aa, Artificial Sequence
  Human antibody library
RASQGISSWLA

SEQ ID NO: 174 - protein, 7 aa, Artificial Sequence
  Human antibody library
AASSLQS

SEQ ID NO: 175 - protein, 9 aa, Artificial Sequence
  Human antibody library
QQTNSFPLT

SEQ ID NO: 176 - protein, 108 aa, Artificial Sequence
  Human antibody library
DIQMTQSPSSVSASVGDRVTITCRASQGISSWLAWYQQKPGKAPKLLIYAASSLQSGVPSRFSGSGSGTDFTLTISSLQPEDFATYYCQQTNSFPLTFGGGTKVDIKR

SEQ ID NO: 177 - protein, 11 aa, Artificial Sequence
  Human antibody library
RASQGISIWLA

SEQ ID NO: 178 - protein, 7 aa, Artificial Sequence
  Human antibody library
GASSLQS

SEQ ID NO: 179 - protein, 9 aa, Artificial Sequence
  Human antibody library
QQANSFPLT

SEQ ID NO: 180 - protein, 108 aa, Artificial Sequence
  Human antibody library
DIQMTQSPSSVSASIGDRVTITCRASQGISIWLAWYQQKPGKAPKLLIYGASSLQSGAPSRFSGSGSGTDFTLTISSLQPEDFATYYCQQANSFPLTFGGGTKVEIKR

SEQ ID NO: 181 - protein, 11 aa, Artificial Sequence
  Human antibody library
RASQGISSWLA

SEQ ID NO: 182 - protein, 7 aa, Artificial Sequence
  Human antibody library
AASSLQS

SEQ ID NO: 183 - protein, 9 aa, Artificial Sequence
  Human antibody library
QQTKSFPIT

SEQ ID NO: 184 - protein, 108 aa, Artificial Sequence
  Human antibody library
DIQMTQSPSSVSASVGDRLTITCRASQGISSWLAWYQQKPGKAPKLLIYAASSLQSGVPSRFSGSGSGTDFTLTISSLQPEDFATYYCQQTKSFPITFGLGTTVDIKR

SEQ ID NO: 185 - protein, 11 aa, Artificial Sequence
  Human antibody library
RASQSVSSWLA

SEQ ID NO: 186 - protein, 7 aa, Artificial Sequence
  Human antibody library
AASSLQS

SEQ ID NO: 187 - protein, 9 aa, Artificial Sequence
  Human antibody library
QQTNSFPLT

SEQ ID NO: 188 - protein, 108 aa, Artificial Sequence
  Human antibody library
DIQMTQSPSSVSASVGDRVTITCRASQSVSSWLAWYQQKPGKAPNLLIYAASSLQSGVPSRFSGSGSGTDFTLTISSLQPEDFATYYCQQTNSFPLTFGQGTRLEIKR

SEQ ID NO: 189 - protein, 11 aa, Artificial Sequence
  Human antibody library
RASQSIDSWLA

SEQ ID NO: 190 - protein, 7 aa, Artificial Sequence
  Human antibody library
AASSLQS

SEQ ID NO: 191 - protein, 9 aa, Artificial Sequence
  Human antibody library
QQTITFPLT

SEQ ID NO: 192 - protein, 108 aa, Artificial Sequence
  Human antibody library
DIQMTQSPSTLSASVGDRVTITCRASQSIDSWLAWYQQKPGKAPKLLIYAASSLQSGVPSRFAGSGSGTDYTLTISSLQPEDFATYYCQQTITFPLTFGGGTKVEIKR

SEQ ID NO: 193 - protein, 11 aa, Artificial Sequence
  Human antibody library
RASQSISSWLA

SEQ ID NO: 194 - protein, 7 aa, Artificial Sequence
  Human antibody library
AASSLQS

SEQ ID NO: 195 - protein, 9 aa, Artificial Sequence
  Human antibody library
QQTITFPLT

SEQ ID NO: 196 - protein, 108 aa, Artificial Sequence
  Human antibody library
DIQMTQSPATLSASVGDRVTITCRASQSISSWLAWYQQKPGKAPKLLIYAASSLQSGVPSRFSGSGSGTDFTLTISSLQPEDFATYYCQQTITFPLTFGGGTKVEIKR

SEQ ID NO: 197 - protein, 11 aa, Artificial Sequence
  Human antibody library
RASQGISDWLA

SEQ ID NO: 198 - protein, 7 aa, Artificial Sequence
  Human antibody library
AASSLQS

SEQ ID NO: 199 - protein, 9 aa, Artificial Sequence
  Human antibody library
QQTITFPLT

SEQ ID NO: 200 - protein, 108 aa, Artificial Sequence
  Human antibody library
DIQMTQSPSSVSASVGDTVTITCRASQGISDWLAWYQQKPGKAPNLLIYAASSLQSGVPSRFSGSKSGTDFTLTISSLQPEDFATYYCQQTITFPLTFGGGTKVEIKR

SEQ ID NO: 201 - protein, 11 aa, Artificial Sequence
  Human antibody library
RASQGIDNWLA

SEQ ID NO: 202 - protein, 7 aa, Artificial Sequence
  Human antibody library
GASSLQS

SEQ ID NO: 203 - protein, 9 aa, Artificial Sequence
  Human antibody library
QQGNTFPLT

SEQ ID NO: 204 - protein, 108 aa, Artificial Sequence
  Human antibody library
DIQMTQSPSSVSASVGDRVTITCRASQGIDNWLAWYQQKPGKAPKLLIYGASSLQSGVPSRFSGSGSGTDYILTINNLQPEDFATYFCQQGNTFPLTFGGGTKVEIKR

SEQ ID NO: 205 - protein, 11 aa, Artificial Sequence
  Human antibody library
RASQDINNWLA

SEQ ID NO: 206 - protein, 7 aa, Artificial Sequence
  Human antibody library
DASSLQT

SEQ ID NO: 207 - protein, 9 aa, Artificial Sequence
  Human antibody library
QQGNSFPLT

SEQ ID NO: 208 - protein, 108 aa, Artificial Sequence
  Human antibody library
DIQMTQSPSSVSASVGDRVTITCRASQDINNWLAWYQQKPGKAPKLLIYDASSLQTGVPSRFSGSGSGTDFTLTISSLQPEDVATYYCQQGNSFPLTFGPGTKVEIKR

SEQ ID NO: 209 - protein, 11 aa, Artificial Sequence
  Human antibody library
RASQGIAGWLA

SEQ ID NO: 210 - protein, 7 aa, Artificial Sequence
  Human antibody library
AASSLQS

SEQ ID NO: 211 - protein, 9 aa, Artificial Sequence
  Human antibody library
QQSYNFPLT

SEQ ID NO: 212 - protein, 107 aa, Artificial Sequence
  Human antibody library
DIQMTSPSSLSASVGDRVTITCRASQGIAGWLAWYQQKPGKAPKLLIYAASSLQSGVPSRFSGSASGTDFTLTISSLQPEDFATYYCQQSYNFPLTFGQGTRVEIKR

SEQ ID NO: 213 - protein, 11 aa, Artificial Sequence
  Human antibody library
GGDNIGTLSVH

SEQ ID NO: 214 - protein, 7 aa, Artificial Sequence
  Human antibody library
DDADRPS

SEQ ID NO: 215 - protein, 11 aa, Artificial Sequence
  Human antibody library
QVWDYTDGLVI

SEQ ID NO: 216 - protein, 111 aa, Artificial Sequence
  Human antibody library
QSALTQPPSVAVAPGQTATITCGGDNIGTLSVHWYRLRPGQAPLLVVYDDADRPSGTPERFSGSNSGNTATLTISRAEAGDEADYYCQVWDYTDGLVIFGGGTTLTVLGQP

SEQ ID NO: 217 - protein, 11 aa, Artificial Sequence
  Human antibody library
GGDSVGITNVH

SEQ ID NO: 218 - protein, 7 aa, Artificial Sequence
  Human antibody library
DDYRRPS

SEQ ID NO: 219 - protein, 11 aa, Artificial Sequence
  Human antibody library
QVWDFSSDLVV

SEQ ID NO: 220 - protein, 111 aa, Artificial Sequence
  Human antibody library
QYELTQTPSVSVAPGQTARITCGGDSVGITNVHWYQQKSGQAPVLVVYDDYRRPSGLPERFSGSNSGNAATLTISRVEAGDEADYYCQVWDFSSDLVVFGGGTKLTVLSQP

SEQ ID NO: 221 - protein, 11 aa, Artificial Sequence
  Human antibody library
QGDSLRSYYAS

SEQ ID NO: 222 - protein, 7 aa, Artificial Sequence
  Human antibody library
HDSVRPS

SEQ ID NO: 223 - protein, 11 aa, Artificial Sequence
  Human antibody library
QVWDLSTDHSV

SEQ ID NO: 224 - protein, 111 aa, Artificial Sequence
  Human antibody library
QSVLTQDPAVSVALGQTVRITCQGDSLRSYYASWYQQQPGQAPLLVVYHDSVRPSGVPERFSGSNSGNTATLTISGVEAGDEADYHCQVWDLSTDHSVFGSGTKVIVLGQP

SEQ ID NO: 225 - protein, 11 aa, Artificial Sequence
  Human antibody library
SGDKLGDKYAF

SEQ ID NO: 226 - protein, 7 aa, Artificial Sequence
  Human antibody library
HDSTRPS

SEQ ID NO: 227 - protein, 10 aa, Artificial Sequence
  Human antibody library
QAWDSSTLYV

SEQ ID NO: 228 - protein, 110 aa, Artificial Sequence
  Human antibody library
QYELTQPPSVSVSPGQTASITCSGDKLGDKYAFWYQQKPGQSPVMVIYHDSTRPSGIPERFSGSNSGNTATLTISGTQAMDEADYYCQAWDSSTLYVFGTGTKVTVLSQP

SEQ ID NO: 229 - protein, 11 aa, Artificial Sequence
  Human antibody library
SGDALPNQYAY

SEQ ID NO: 230 - protein, 7 aa, Artificial Sequence
  Human antibody library
KDKERPL

SEQ ID NO: 231 - protein, 11 aa, Artificial Sequence
  Human antibody library
QSADTNDAYKV

SEQ ID NO: 232 - protein, 111 aa, Artificial Sequence
  Human antibody library
QSELTQPPSVSVAPGQTARITCSGDALPNQYAYWYQQKSGQAPVLLLYKDKERPLGIPERFSGSSSGTTVTLTISGVQAEDEADYYCQSADTNDAYKVFGGGTKLTVLGQP

SEQ ID NO: 233 - protein, 11 aa, Artificial Sequence
  Human antibody library
SGDELGDKYVS

SEQ ID NO: 234 - protein, 7 aa, Artificial Sequence
  Human antibody library
HDTERPS

SEQ ID NO: 235 - protein, 11 aa, Artificial Sequence
  Human antibody library
QSANNFTTYYA

SEQ ID NO: 236 - protein, 111 aa, Artificial Sequence
  Human antibody library
QSVLTQPPSVSVSPGQTANIICSGDELGDKYVSWYQQKPGQAPVMVIYHDTERPSGIPERFSGSSSGTTVTLTISGVQAEDEADYYCQSANNFTTYYAFGPGTKVTVLSQP

SEQ ID NO: 237 - protein, 11 aa, Artificial Sequence
  Human antibody library
SGNALSRQYAY

SEQ ID NO: 238 - protein, 7 aa, Artificial Sequence
  Human antibody library
KDSERSS

SEQ ID NO: 239 - protein, 11 aa, Artificial Sequence
  Human antibody library
QSADITDTYYV

SEQ ID NO: 240 - protein, 111 aa, Artificial Sequence
  Human antibody library
QYELTQPPSVSVSPGQTASITCSGNALSRQYAYWYQQKTGQAPVIVIYKDSERSSGIPERFSGSSSGTTVTLTISGVQAEDEADYYCQSADITDTYYVFGTGTKVTVLGQL

SEQ ID NO: 241 - protein, 11 aa, Artificial Sequence
  Human antibody library
GGNNIGSLSVH

SEQ ID NO: 242 - protein, 7 aa, Artificial Sequence
  Human antibody library
DDGDRPS

SEQ ID NO: 243 - protein, 11 aa, Artificial Sequence
  Human antibody library
QVWDYSDDLVV

SEQ ID NO: 244 - protein, 111 aa, Artificial Sequence
  Human antibody library
  misc_feature (102)..(102): Xaa can be any naturally occurring amino acid
QSELTQPPSVSVAPGQTARINCGGNNIGSLSVHWYQQKPGQAPALVVYDDGDRPSGIPERFSGSNSGNTATLTIVRAEAGDEADYYCQVWDYSDDLVVFGGXTTLTVVGQP

SEQ ID NO: 245 - protein, 11 aa, Artificial Sequence
  Human antibody library
SGERLGDKYVF

SEQ ID NO: 246 - protein, 7 aa, Artificial Sequence
  Human antibody library
QDYKRPS

SEQ ID NO: 247 - protein, 10 aa, Artificial Sequence
  Human antibody library
QVWDMTTLVV

SEQ ID NO: 248 - protein, 110 aa, Artificial Sequence
  Human antibody library
QYELTQPPSLSVPPGQTASITCSGERLGDKYVFWYQRKAGQSPVLVIYQDYKRPSGIPERFSGSNSGNTATLTISGTQAMDEAEYICQVWDMTTLVVFGGGTKLTVLGQP

(19) United States
(12) Patent Application Publication
(10) Pub. No.: US 2018/0244779 A1
(43) Pub. Date: Aug. 30, 2018

(54) IMMUNOMODULATORY AGENTS

(71) Applicant: KADMON CORPORATION LLC, New York, NY (US)

(72) Inventors: Dan Lu, Montvale, NJ (US); Zhenping Zhu, Woodcliff Lake, NJ (US)

(21) Appl. No.: 15/549,016

(22) PCT Filed: Feb. 8, 2016
(86) PCT No.: PCT/US16/17021

§ 371 Date: Aug. 4, 2017

Related U.S. Application Data
(60) Provisional application No. 62/113,132, filed on Feb. 6, 2015.

(51) Int. Cl. C07K 16/28

(52) U.S. Cl. CPC C07K 16/2818; C07K 2317/92; C07K 2317/76; A61K 39/3955; C07K 2317/55; C07K 2317/33; C07K 2317/56

(57) ABSTRACT

The invention provides antibodies that specifically bind to PD-1, nucleic acid molecules encoding the same, and therapeutic compositions thereof. The agents inhibit PD-1-mediated immunosuppression and enhance cell and cytokine mediated immunity for the treatment of neoplastic and infectious diseases.

### CROSS-REFERENCE TO RELATED APPLICATIONS

This application claims priority to U.S. Provisional Application No. 62/113,132, filed Feb. 6, 2015, the content of which is incorporated herein by reference in its entirety.

### FIELD OF THE INVENTION

The invention provides monoclonal antibodies that specifically bind to PD-1 and therapeutic compositions thereof. The agents enhance T cell and NK cell function to increase cell and cytokine mediated immunity for the treatment of various immune dysfunction related disorders including cancers and infectious diseases.

### BACKGROUND OF THE INVENTION

Programmed death 1 (PD-1) is a member of the CD28 family of receptors comprising CD28, CTLA-4, PD-1, ICOS, and BTLA (Freeman et al. (2000) J Exp Med 192:1027-34; Latchman et al. (2001) Nat Immunol 2:261-8). PD-1 is an inducible immunosuppressive receptor mainly upregulated on activated T cells and B cells during the progression of immunopathological conditions. PD-1 interaction with its ligand PD-L1 results in the inhibition of TCR and BCR mediated proliferation and cytokine production and induction of apoptosis of antigen specific T cells through the intrinsic PD-1 mediated negative signaling of an immunoreceptor tyrosine-based inhibitory motif (ITIM) (Agata et al. (1996) Int. Immunol. 8:765, Unkeless and Jin. (1997) Curr. Opin. Immunol. 9:338-343, Okzaki et al. (2001) PNAS 98:13866-71, Dong et al. (2002) Nat. Med. 8:793-800). PD-L1 is a cell surface glycoprotein and a major ligand for PD-1. PD-L1 is also inducible on lymphoid tissues and non-lymphoid peripheral tissues following cellular activation. PD-L1 is upregulated in a variety of affected cell types including cancer and stromal cells in addition to immune cells, and plays an active role in immunosuppression during the course of the deterioration of diseases (Iwai et al (2002) PNAS 99:12293-7, Ohigashi et al. (2005) Clin Cancer Res 11:2947-53). PD-L1 upregulation has been linked to poor clinical outcomes in a variety of cancers and viral infection (Hofmeyer et al. (2011) J. BioMed. Biotech. 2011:1-9, McDermott and Atkins. (2013) Cancer Med. 2:662-73). The blockade of PD-1 or PD-L1 by antibody promoted CD8 T cell infiltration, CTL activity and increased presence of Th1 cytokine IFN-gamma in preclinical and clinical settings (Zhou et al. (2010) J. Immunol. 185:5082-92, Nomi et al. (2007) Clin Cancer Res. 13:2152-7, Flies et al. (2011) Yale J. Bio. Med. 48:409-21, Zitvogel and Kroemer. (2012) Oncolmmunol. 1:1223-25).

The PD-1 antibodies of the present invention are used as an immunomodulating agent and may be efficacious when used as monotherapy or when combined with antibodies to other immunosuppressive molecules.

### SUMMARY OF THE INVENTION

The present invention provides antibodies and binding proteins that bind to PD-1. In certain embodiments of the invention, the antibodies bind to PD-1 and block interaction with PD-L1. By blocking the interaction of PD-1 with PD-L1, such antibodies are useful to reduce or inhibit immunosuppression.

In one embodiment, the invention provides an antibody or fragment that binds to PD-1, which comprises a heavy chain variable domain which comprises SEQ ID NO: 6, SEQ ID NO: 16, SEQ ID NO: 26, or SEQ ID NO: 36. In such embodiments, the heavy chain variable domain is at least 80%, or at least 85%, or at least 90%, or at least 95% identical to SEQ ID NO: 6, SEQ ID NO: 16, SEQ ID NO: 26, SEQ ID NO: 36. The antibodies may further comprise a light chain variable domain which comprises SEQ ID NO: 10, SEQ ID NO: 20, SEQ ID NO: 30, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116, SEQ ID NO: 120, SEQ ID NO: 124, SEQ ID NO: 128, SEQ ID NO: 132, SEQ ID NO: 136, SEQ ID NO: 140, SEQ ID NO: 144, SEQ ID NO: 148, SEQ ID NO: 152, SEQ ID NO: 156, SEQ ID NO: 160, SEQ ID NO: 164, SEQ ID NO: 168, SEQ ID NO: 172, SEQ ID NO: 176, SEQ ID NO: 180, SEQ ID NO: 184, SEQ ID NO: 188, SEQ ID NO: 192, SEQ ID NO: 196, SEQ ID NO: 200, SEQ ID NO: 204, SEQ ID NO: 208, SEQ ID NO: 212, SEQ ID NO: 216, SEQ ID NO: 220, SEQ ID NO: 224, SEQ ID NO: 228, SEQ ID NO: 232, SEQ ID NO: 236, SEQ ID NO: 240, SEQ ID NO: 244, or SEQ ID NO: 248. In some such embodiments the light chain variable domain is at least 80%, or at least 85%, or at least 90%, or at least 95% identical to SEQ ID NO: 10, SEQ ID NO: 20, SEQ ID NO: 30, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116, SEQ ID NO: 120, SEQ ID NO: 124, SEQ ID NO: 128, SEQ ID NO: 132, SEQ ID NO: 136, SEQ ID NO: 140, SEQ ID NO: 144, SEQ ID NO: 148, SEQ ID NO: 152, SEQ ID NO: 156, SEQ ID NO: 160, SEQ ID NO: 164, SEQ ID NO: 168, SEQ ID NO: 172, SEQ ID NO: 176, SEQ ID NO: 180, SEQ ID NO: 184, SEQ ID NO: 188, SEQ ID NO: 192, SEQ ID NO: 196, SEQ ID NO: 200, SEQ ID NO: 204, SEQ ID NO: 208, SEQ ID NO: 212, SEQ ID NO: 216, SEQ ID NO: 220, SEQ ID NO: 224, SEQ ID NO: 228, SEQ ID NO: 232, SEQ ID NO: 236, SEQ ID NO: 240, SEQ ID NO: 244, or SEQ ID NO: 248.

In another embodiment, the invention provides an antibody or fragment thereof that binds to PD-1, wherein the heavy chain comprises a CDR-1H (Kabat) which has SEQ ID NO: 1, SEQ ID NO: 11, SEQ ID NO: 21, or SEQ ID NO: 31, a CDR-2H (Kabat) which has SEQ ID NO: 3, SEQ ID NO: 13, SEQ ID NO: 23, or SEQ ID NO: 33, and a CDR-3H which has SEQ ID NO: 5, SEQ ID NO: 15, SEQ ID NO: 25, or SEQ ID NO: 35. In another embodiment, the invention provides an antibody or fragment thereof that binds to PD-1, wherein the heavy chain comprises a CDR-1H (Chothia) which has SEQ ID NO: 2, SEQ ID NO: 12, SEQ ID NO: 22, or SEQ ID NO: 32, a CDR-2H (Chothia) which has SEQ ID NO: 4, SEQ ID NO: 14, SEQ ID NO: 24, or SEQ ID NO: 34, and a CDR-3H which has SEQ ID NO: 5, SEQ ID NO: 15, SEQ ID NO: 25, or SEQ ID NO: 35. In another embodiment, the invention provides an antibody or fragment thereof that binds to PD-1, wherein the light chain comprises a CDR-1L which has SEQ ID NO: 7, SEQ ID NO: 17, SEQ ID NO: 27, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, SEQ ID NO: 89, SEQ ID NO: 93, SEQ ID NO: 97, SEQ ID NO: 101, SEQ ID NO: 105, SEQ ID NO: 109SEQ ID NO: 113, SEQ ID NO: 117, SEQ ID NO: 121, SEQ ID NO: 125, SEQ ID NO: 129, SEQ ID NO: 133, SEQ ID NO: 137, SEQ ID NO: 141, SEQ ID NO: 145, SEQ ID NO: 149, SEQ ID NO: 153, SEQ ID NO: 157, SEQ ID NO: 161, SEQ ID NO: 165, SEQ ID NO: 169, SEQ ID NO: 173, SEQ ID NO: 177, SEQ ID NO: 181, SEQ ID NO: 185, SEQ ID NO: 189, SEQ ID NO: 193, SEQ ID NO: 197, SEQ ID NO: 201, SEQ ID NO: 205, SEQ ID NO: 209, SEQ ID NO: 213, SEQ ID NO: 217, SEQ ID NO: 221, SEQ ID NO: 225, SEQ ID NO: 229, SEQ ID NO: 233, SEQ ID NO: 237, SEQ ID NO: 241, or SEQ ID NO: 245, a CDR-2L which has SEQ ID NO: 8, SEQ ID NO: 18, SEQ ID NO: 28, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90, SEQ ID NO: 94, SEQ ID NO: 98, SEQ ID NO: 102, SEQ ID NO: 106, SEQ ID NO: 110, SEQ ID NO: 114, SEQ ID NO: 118, SEQ ID NO: 122, SEQ ID NO: 126, SEQ ID NO: 130, SEQ ID NO: 134, SEQ ID NO: 138, SEQ ID NO: 142, SEQ ID NO: 146, SEQ ID NO: 150, SEQ ID NO: 154, SEQ ID NO: 158, SEQ ID NO: 162, SEQ ID NO: 166, SEQ ID NO: 170, SEQ ID NO: 174, SEQ ID NO: 178, SEQ ID NO: 182, SEQ ID NO: 186, SEQ ID NO: 190, SEQ ID NO: 194, SEQ ID NO: 198, SEQ ID NO: 202, SEQ ID NO: 206, SEQ ID NO: 210, SEQ ID NO: 214, SEQ ID NO: 218, SEQ ID NO: 222, SEQ ID NO: 226, SEQ ID NO: 230, SEQ ID NO: 234, SEQ ID NO: 238, SEQ ID NO: 242, or SEQ ID NO: 246, and a CDR-3L which has SEQ ID NO: 9, SEQ ID NO: 19, SEQ ID NO: 29, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, SEQ ID NO: 91, SEQ ID NO: 95, SEQ ID NO: 99, SEQ ID NO: 103, SEQ ID NO: 107, SEQ ID NO: 111, SEQ ID NO: 115, SEQ ID NO: 119, SEQ ID NO: 123, SEQ ID NO: 127, SEQ ID NO: 131, SEQ ID NO: 135, SEQ ID NO: 139, SEQ ID NO: 143, SEQ ID NO: 147, SEQ ID NO: 151, SEQ ID NO: 155, SEQ ID NO: 159, SEQ ID NO: 163, SEQ ID NO: 167, SEQ ID NO: 171, SEQ ID NO: 175, SEQ ID NO: 179, SEQ ID NO: 183, SEQ ID NO: 187, SEQ ID NO: 191, SEQ ID NO: 195, SEQ ID NO: 199, SEQ ID NO: 203, SEQ ID NO: 207, SEQ ID NO: 211, SEQ ID NO: 215, SEQ ID NO: 219, SEQ ID NO: 223, SEQ ID NO: 227, SEQ ID NO: 231, SEQ ID NO: 235, SEQ ID NO: 239, SEQ ID NO: 243, SEQ ID NO: 247.

The invention also provides conjugates of the antibodies, for example, and without limitation, to imaging agents, therapeutic agents, or cytotoxic agents.

The invention further provides compositions comprising the antibodies and conjugates and a pharamaceutically acceptable carrier.

The invention provides a method of inhibiting the interaction of PD-1 with PD-L1 in a subject, which comprises administering an effective amount of an antibody or fragment of the invention. The invention further provides a method of inhibiting immunosuppression mediated by PD-1 which comprises administering an effective amount of the antibody or fragment of the invention.

The invention further provides a method of stimulating an immune response against a cell or tissue that expresses PD-1, which comprises administering to a subject an effective amount of the antibody or fragment of the invention. In certain embodiments, the cell or tissue that expresses PD-1 is a neoplastic cell or an infected cell.

### BRIEF DESCRIPTION OF THE FIGURES

FIG. 1 depicts binding to anti-hFc of antibodies R3A1, R3A2, R4B3, R3B7, and R3D6.

FIG. 2 depicts binding to EGFR-Fc of antibodies R3A1, R3A2, R4B3, R3B7, and R3D6.

FIG. 3 depicts binding to ckit-Fc of antibodies R3A1, R3A2, R4B3, R3B7, and R3D6.

FIG. 4 depicts binding to KDR-Fc of antibodies R3A1, R3A2, R4B3, R3B7, and R3D6.

FIG. 5 depicts binding to hPD-1-Fc of antibodies R3A1, R3A2, R4B3, R3B7, and R3D6.

FIG. 6 depicts blocking of PD-L1 to hPD-1 by antibodies R3A1, R3A2, R4B3, R3B7, and R3D6.

FIG. 7 depicts binding to mPD-1-Fc of antibodies R3A1, R3A2, R4B3, R3B7, and R3D6.

FIG. 8 depicts binding to PD-1-Fc of antibodies A2_#1 and A2_#2.

FIG. 9 depicts blocking of PD-L1 to hPD-1 by antibodies A2_#1 and A2_#2.

FIG. 10 depicts blocking of PD-L2 to hPD-1 by antibodies A2_#1 and A2_#2.

FIG. 11 depicts binding of activity to hPD-1 expressing HEK293 cells as measured by flow cytometry of antibodies A2_#1 and A2_#2.

FIG. 12 depicts binding activity to hPD-1 expressing CD4 T cells as measured by flow cytometry of antibodies A2_#1 and A2_#2.

FIG. 13 depicts binding activity to hPD-1 expressing CD8 T cells as measured by flow cytometry of antibodies A2_#1 and A2_#2.

FIG. 14 depicts IL2 secretion in SEB stimulated PBMC cultured with antibodies A2 #1 and A2 #2.

FIG. 15 depicts INFγ secretion in SEB stimulated PBMC cultured with antibodies A2 #1 and A2 #2.

FIG. 16 depicts the proliferation of CD4 T cells cultured with antibodies A2 #1 and A2 #2.

FIG. 17 depicts INFγ secretion in Mixed Lymphocyte reaction cultured with antibodies A2 #1 and A2 #2.

### DETAILED DESCRIPTION

The interaction of PD-1 on immune cells with PD-L1 inhibits proliferation and cytokine production by immune cells. PD-L1 is also inducible and upregulated in various tissues, including cancer. Together, PD-1 and PD-L1 play a role in immunosuppression. The invention provides novel antibodies or antigen binding fragments of such antibodies that bind to PD-1 and block the interaction with PD-L1. In embodiments of the invention, the antibodies reduce or inhibit immunosuppression.

Novel antibodies of the invention are set forth in Table 1 and the accompanying sequence listing, which set forth amino acid sequences of heavy and light chain CDRs (identified according to the identification systems of Kabat and Chothia), as well as complete heavy and light chain variable region. The first two heavy chain CDRs are identified according to the common systems of Kabat and Chothia, which provide distinct, but overlapping locations for the CDRs. A comparison of the numerous heavy and light chains shows a significant similarity among many of the CDR sequences. Accordingly, it would be expected that many of the CDRs can be mixed and matched among the sequences.

The antibodies can have one or more amino acid substitutions, deletions, insertions, and/or additions. In certain embodiments, the antibodies comprise one of the above-mentioned heavy chain variable domains and one of the above-mentioned light chain variable domains. In certain embodiments, the PD-1 antibodies or binding fragments thereof comprise one or more CDRs or one or more variable domains with an amino acid sequence at least 85% at least 90%, at least 95%, at least 97%, at least 98%, or at least 99%, identical to the CDR and variable domain sequences set forth in Table 1.

“Identity” refers to the number or percentage of identical positions shared by two amino acid or nucleic acid sequences, taking into account the number of gaps, and the length of each gap, which need to be introduced for optimal alignment of the two sequences. “Substantially identical” means an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Preferably, the amino acid sequence is at least 80%, or at least 85%, or at least 90%, or at least 95% identical to an amino acid sequence disclosed herein. Methods and computer programs for determining sequence similarity are publically available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Antibodies of the invention also include those for which binding characteristics have been improved by direct mutation, methods of affinity maturation, phage display, or chain shuffling. Affinity and specificity may be modified or improved by mutating CDRs and screening for antigen binding sites having the desired characteristics. CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Biol., 226: 889-896 (1992)). For example, phage display vectors containing heavy and light chain variable region genes may be propagated in mutator strains of E. coli (see, e.g., Low et al., J. Mol. Biol., 250: 359-368 (1996)). These methods of mutagenesis are illustrative of the many methods known to one of skill in the art.

To minimize the immunogenicity, antibodies which comprise human constant domain sequences are preferred. The antibodies may be or may combine members of any immunoglobulin class, such as IgG, IgM, IgA, IgD, or IgE, and the subclasses thereof. The antibody class may be selected to optimize effector functions (e.g., complement dependent cytotoxicity (CDC) and antibody dependent cellular cytotoxicity (ADCC)) of natural antibodies.

Certain embodiments of the invention involve the use of PD-1-binding antibody fragments. An Fv is the smallest fragment that contains a complete heavy and light chain variable domain, including all six hypervariable loops (CDRs). Lacking constant domains, the variable domains are noncovalently associated. The heavy and light chains may be connected into a single polypeptide chain (a “single-chain Fv” or “scFv”) using a linker that allows the V_{H} and V_{L} domains to associate to form an antigen binding site. In an embodiment of the invention, the linker is (Gly-Gly-Gly-Gly-Ser)₃. Since scFv fragments lack the constant domains of whole antibodies, they are considerably smaller than whole antibodies. scFv fragments are also free of normal heavy-chain constant domain interactions with other biological molecules which may be undesired in certain embodiments.

Fragments of an antibody containing V_{H}, V_{L}, and optionally C_{L}, C_{H}1, or other constant domains can also be used. Monovalent fragments of antibodies generated by papain digestion are referred to as Fab and lack the heavy chain hinge region. Fragments generated by pepsin digestion, referred to as F(ab′)₂, retain the heavy chain hinge and are divalent. Such fragments may also be recombinantly produced. Many other useful antigen-binding antibody fragments are known in the art, and include, without limitation, diabodies, triabodies, single domain antibodies, and other monovalent and multivalent forms.

The invention further provides multivalent antigen-binding proteins, which can be in the form, without limitation, of antibodies, antigen-binding fragments thereof, and proteins comprising all or part of antigen-binding portions of antibodies. Multivalent antigen-binding proteins may be monospecific, bispecific, or multispecific. The term specificity refers to the number of different types of antigenic determinants to which a particular molecule can bind. If an immunoglobulin molecule binds to only one type of antigenic determinant, the immunoglobulin molecule is monospecific. If the immunoglobulin molecule binds to different types of antigenic determinants then the immunoglobulin molecule is multispecific.

In an embodiment of the invention, the PD-1 binding protein has an on rate constant (Kon) of at least about 10² M⁻¹s⁻¹; at least about 10³ M⁻¹s⁻¹; at least about 10⁴ M⁻¹s⁻¹; at least about 10⁵ M⁻¹s⁻¹; or at least about 10⁶ M⁻¹s⁻¹, as measured by surface plasmon resonance. In an embodiment, the PD-L1 binding protein has an on rate constant (Kon) between 10² M⁻¹s⁻¹ and 10³ M⁻¹s⁻¹; between 10³ M⁻¹s⁻¹ and 10⁴ M⁻¹s⁻¹; between 10⁴ M⁻¹s⁻¹ and 10⁵ M⁻¹s⁻¹; or between 10⁵ M⁻¹s⁻¹ and 10⁶ M⁻¹s⁻¹, as measured by surface plasmon resonance.

In another embodiment the PD-1 binding protein has an off rate constant (Koff) of at most about 10⁻³s⁻¹; at most about 10⁻⁴s⁻¹; at most about 10⁻⁵s⁻¹; or at most about 10⁻⁶s⁻¹, as measured by surface plasmon resonance. In an embodiment, the PD-L1 binding protein has an off rate constant (Koff) of 10⁻³s⁻¹ to 10⁻⁴s⁻¹; of 10⁻⁴s⁻¹ to 10⁻⁵s⁻¹; or of 10⁻⁵s⁻¹ to 10⁻⁶s⁻¹, as measured by surface plasmon resonance.

In another embodiment the PD-1 binding protein has a dissociation constant (K_{D}) of at most about 10⁻⁷ M; at most about 10⁻⁸ M; at most about 10⁻⁹ M; at most about 10⁻¹⁰ M; at most about 10⁻¹¹ M; at most about 10⁻¹² M; or at most 10⁻¹³ M. In an embodiment, the binding protein has a dissociation constant (K_{D}) to its targets of 10⁻⁷ M to 10⁻⁸ M; of 10⁻⁸ M to 10⁻⁹ M; of 10⁻⁹ M to 10⁻¹⁰ M; of 10⁻¹⁰ M to 10⁻¹¹ M; of 10⁻¹¹ M to 10⁻¹² M; or of 10⁻¹² M to 10⁻¹³ M.

The binding protein described herein may be a conjugate further comprising an imaging agent, a therapeutic agent, or a cytotoxic agent. In an embodiment, the imaging agent is a radiolabel, an enzyme, a fluorescent label, a luminescent label, a bioluminescent label, a magnetic label, or biotin. In another embodiment, the radiolabel is: ³H, ¹⁴C, ³⁵S, ⁹⁰ Y, ⁹⁹Tc, ¹¹¹In, ¹²⁵In, ¹³¹I, ¹⁷⁷Lu, or ¹⁵³Sm. In yet another embodiment, the therapeutic or cytotoxic agent is an anti-metabolite, an alkylating agent, an antibiotic, a growth factor, a cytokine, an anti-angiogenic agent, an anti-mitotic agent, an anthracycline, toxin, or an apoptotic agent. As discussed below, immunostimulatory cytokines are of particular importance.

As exemplified herein, the PD-1-binding portion of the molecule is an antigen-binding domain of an antibody. Several novel antibody heavy and light chain variable domains and antibodies that include them are provided. According to the invention, the PD-1-binding portion can be any agent that binds to PD-1 and blocks immunosuppression. These include anti-PD-1 antibodies and fragments, not limited to those novel antibodies disclosed herein, as well as peptides and proteins derived from PD-L1, the natural ligand of PD-1.

It is understood that the anti-PD-1 antibodies of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, sucrose, polysorbate, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibodies.

In the methods of the present invention, a therapeutically effective amount of an antibody or antibody fragment, of the invention is administered to a mammal in need thereof. The term “administering” as used herein means delivering the antibodies of the present invention to a mammal by any method that may achieve the result sought. They may be administered, for example, intravenously or intramuscularly. Although the exemplified antibodies of the invention are particularly useful for administration to humans, they may be administered to other mammals as well. The term “mammal” as used herein is intended to include, but is not limited to, humans, laboratory animals, domestic pets and farm animals. “Therapeutically effective amount” means an amount of antibody of the present invention that, when administered to a mammal, is effective in producing the desired therapeutic effect, such as inhibiting kinase activity.

Antibodies of the invention are useful for inhibiting tumors and other neoplastic diseases, as well as treating other pathologic conditions associated with immunosuppression. Tumors that can be treated include primary tumors, metastatic tumors, and refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued. The antibodies are effective for treating vascularized tumors and tumor that are not vascularized, or not yet substantially vascularized.

Examples of solid tumors which may be accordingly treated include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Examples of vascularized skin cancers for which the antagonists of this invention are effective include squamous cell carcinoma, basal cell carcinoma and skin cancers that can be treated by suppressing the growth of malignant keratinocytes, such as human malignant keratinocytes.

Examples of non-solid tumors include leukemia, multiple myeloma and lymphoma. In some embodiments the tumor may be unresponsive to cytokines, such as IL15. Some examples of leukemias include acute myelogenous leukemia (AML), chronic myelogenous leukemia (CML), acute lymphocytic leukemia (ALL), chronic lymphocytic leukemia (CLL), erythrocytic leukemia or monocytic leukemia. Some examples of lymphomas include Hodgkin's and non-Hodgkin's lymphoma.

The PD-1 antibodies of the invention are also used in the treatment of viral infections. PD-1 expression on T cells correlates with viral load in HIV and HCV infected patients and PD-1 expression has been identified as a marker for exhausted virus-specific CD8+ T cells. For example, PD-1⁺CD8⁺ T cells show impaired effector functions and PD-1 associated T cell exhaustion which can be restored by blocking the PD-1/PD-L1 interaction. This results in recovery of virus-specific CD8+ T cell mediated immunity, indicating that interrupting PD-1 signaling using an antagonistic antibody restores T-cell effector functions. Immunotherapy based on the blockade of PD-1/PD-L1 results in breakdown of T-cell tolerance not only to tumor antigens, but also provides a strategy to reactivate virus-specific effector T cells and eradicate pathogens in chronic viral infections. Accordingly, the antibodies of the invention are useful to treat chronic viral infections, including, without limitation, HCV and HIV, and lymphocytic choriomeningitis virus (LCMV).

The antibodies of the invention can be advantageously administered with second agents to patients in need thereof. For example, in some embodiments, an antibody of the invention is administered to a subject with an anti-neoplastic agent. In some embodiments, an antibody of the invention is administered to a subject with an angiogenesis inhibitor. In some embodiments, an antibody of the invention is administered with an anti-inflammatory agent or an immunosuppressant.

Antineoplastic agents include cytotoxic chemotherapeutic agents, targeted small molecules and biological molecules, and radiation. Non-limiting examples of chemotherapeutic agents include cisplatin, dacarbazine (DTIC), dactinomycin, irinotecan, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol and combinations thereof.

Targeted small molecules and biological molecules include, without limitation, inhibitors of components of signal transduction pathways, such as modulators of tyrosine kinases and inhibitors of receptor tyrosine kinases, and agents that bind to tumor-specific antigens. Non-limiting examples of growth factor receptors involved in tumorigenesis are the receptors for platelet-derived growth factor (PDGFR), insulin-like growth factor (IGFR), nerve growth factor (NGFR), and fibroblast growth factor (FGFR), and receptors of the epidermal growth factor receptor family, including epidermal growth factor receptor (EGFR, also known as erbB1), HER2 (erbB2), erbB3, and erbB4.

EGFR antagonists include antibodies that bind to EGFR or to an EGFR ligand, and inhibit ligand binding and/or receptor activation. For example, the agent can block formation of receptor dimers or heterodimer with other EGFR family members. Ligands for EGFR include, for example, EGF, TGF-α amphiregulin, heparin-binding EGF (HB-EGF) and betaregullulin. An EGFR antagonist can bind externally to the extracellular portion of EGFR, which may or may not inhibit binding of the ligand, or internally to the tyrosine kinase domain. EGFR antagonists further include agents that inhibit EGFR-dependent signal transduction, for example, by inhibiting the function of a component of the EGFR signal transduction pathway. Examples of EGFR antagonists that bind EGFR include, without limitation, biological molecules, such as antibodies (and functional equivalents thereof) specific for EGFR, and small molecules, such as synthetic kinase inhibitors that act directly on the cytoplasmic domain of EGFR.

Small molecule and biological inhibitors include inhibitors of EGFR, including gefitinib, erlotinib, and cetuximab, inhibitors of HER2 (e.g., trastuzumab, trastuzumab emtansine (trastuzumab-DM1; T-DM1) and pertuzumab), anti-VEGF antibodies and fragments (e.g., bevacizumab), antibodies that inhibit CD20 (e.g., rituximab, ibritumomab), anti-VEGFR antibodies (e.g., ramucirumab (IMC-1121B), IMC-1C11, and CDP791), anti-PDGFR antibodies, and imatinib. Small molecule kinase inhibitors can be specific for a particular tyrosine kinase or be inhibitors of two or more kinases. For example, the compound N-(3,4-dichloro-2-fluorophenyl)-7-({[(3aR,6aS)-2-methyloctahydrocyclopenta[c]pyrrol-5-yl]methyl}oxy)-6-(methyloxy)quinazolin-4-amine (also known as XL647, EXEL-7647 and KD-019) is an in vitro inhibitor of several receptor tyrosine kinases (RTKs), including EGFR, EphB4, KDR (VEGFR), Flt4 (VEGFR3) and ErbB2, and is also an inhibitor of the SRC kinase, which is involved in pathways that result in nonresponsiveness of tumors to certain TKIs. In an embodiment of the invention, treatment of a subject in need comprises administration of a rho-kinase inhibitor.

Dasatinib (BMS-354825; Bristol-Myers Squibb, New York) is another orally bioavailable, ATP-site competitive Src inhibitor. Dasatanib also targets Bcr-Abl (FDA-approved for use in patients with chronic myelogenous leukemia (CML) or Philadelphia chromosome positive (Ph+) acute lymphoblastic leukemia (ALL)) as well as c-Kit, PDGFR, c-FMS, EphA2, and SFKs. Two other oral tyrosine kinase inhibitor of Src and Bcr-Abl are bosutinib (SKI-606) and saracatinib (AZD0530).

In an embodiment of the invention, a PD-1 antibody of the invention is used in combination with an anti-viral agent to treat a chronic virus infection. For example, to treat HCV, the following agents can be used. HCV protease inhibitors include, without limitation, boceprevir, telaprevir (VX-950), ITMN-191, SCH-900518, TMC-435, BI-201335, MK-7009, VX-500, VX-813, BMS790052, BMS650032, and VBY376. HCV nonstructural protein 4B (NS4B) inhibitors include, but are not limited to, clemizole, and other NS4B-RNA binding inhibitors, including but not limited to benzimidazole RBIs (B-RBIs) and indazole RBIs (I-RBIs). HCV nonstructural protein 5A (NSSA) inhibitors include, but are not limited to, BMS-790052, A-689, A-831, EDP239, GS5885, and PP1461. HCV polymerase (NS5B) inhibitors include, but are not limited to nucleoside analogs (e.g., valopicitabine, R1479, R1626, R7128), nucleotide analogs (e.g., IDX184, PSI-7851, PSI-7977, and non-nucleoside analogs (e.g., filibuvir, HCV-796, VCH-759, VCH-916, ANA598, VCH-222 (VX-222), BI-207127, MK-3281, ABT-072, ABT-333, GS9190, BMS791325). Also, ribavirin or a ribavirin analog such as Taribavirin (viramidine; ICN 3142), Mizoribine, Merimepodib (VX-497), Mycophenolate mofetil, and Mycophenolate can be used.

In certain embodiments, a dose of an antibody of the invention is administered to a subject every day, every other day, every couple of days, every third day, once a week, twice a week, three times a week, or once every two weeks. In other embodiments, two, three or four doses of a compound or a composition is administered to a subject every day, every couple of days, every third day, once a week or once every two weeks. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

Methods of administration include but are not limited to parenteral, intradermal, intravitrial, intramuscular, intraperitoneal, intravenous, subcutaneous, intranasal, epidural, oral, sublingual, intranasal, intracerebral, intravaginal, transdermal, transmucosal, rectally, by inhalation, or topically, particularly to the ears, nose, eyes, or skin. The mode of administration is left to the discretion of the practitioner. In most instances, administration will result in the release of a compound into the bloodstream. For treatment of ocular disease, intravitrial administration of biological agents is preferred.

In specific embodiments, it may be desirable to administer a compound locally. This may be achieved, for example, and not by way of limitation, by local infusion, topical application, by injection, by means of a catheter, or by means of an implant, said implant being of a porous, non-porous, or gelatinous material, including membranes, such as sialastic membranes, or fibers. In such instances, administration may selectively target a local tissue without substantial release of a compound into the bloodstream.

Pulmonary administration can also be employed, e.g., by use of an inhaler or nebulizer, and formulation with an aerosolizing agent, or via perfusion in a fluorocarbon or synthetic pulmonary surfactant. In certain embodiments, a compound is formulated as a suppository, with traditional binders and vehicles such as triglycerides.

In another embodiment, a compound is delivered in a vesicle, in particular a liposome (See Langer, 1990, Science 249:1527-1533; Treat et al., in Liposomes in the Therapy of Infectious Disease and Bacterial infection, Lopez-Berestein and Fidler (eds.), Liss, New York, pp. 353-365 (1989); Lopez Berestein, ibid., pp. 317-327; see generally ibid.).

In another embodiment, a compound is delivered in a controlled release system (See, e.g., Goodson, in Medical Applications of Controlled Release, supra, vol. 2, pp. 115-138 (1984)). Examples of controlled-release systems are discussed in the review by Langer, 1990, Science 249:1527-1533 may be used. In one embodiment, a pump may be used (See Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (See Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; See also Levy et al., 1985, Science 228:190; During et al., 1989, Ann. Neurol. 25:351; Howard et al., 1989, J. Neurosurg. 71:105).

The above-described administration schedules are provided for illustrative purposes only and should not be considered limiting. A person of ordinary skill in the art will readily understand that all doses are within the scope of the invention.

It is to be understood and expected that variations in the principles of invention herein disclosed may be made by one skilled in the art and it is intended that such modifications are to be included within the scope of the present invention.

Throughout this application, various publications are referenced. These publications are hereby incorporated into this application by reference in their entireties to more fully describe the state of the art to which this invention pertains. The following examples further illustrate the invention, but should not be construed to limit the scope of the invention in any way.

### EXAMPLES

Specific High Affinity Antibodies to PD-L1 from Phage-Display Library

Anti-PD-1 antibodies with high affinity were obtained using a phage display library. In one procedure, phage Fabs amplified from Dyax libraries were panned on soluble PD-1-Fc captured by biotin-anti-hFc Ab and magnetic strep-beads.

In a second procedure, phage Fabs amplified from the Dyax libraries were panned on tube immobilized PD-1-Fc.

In a third procedure, phage Fabs amplified from the Dyax libraries were panned on tube immobilized PD-1-Fc for the first round, and then panned on PD-1 transfected 293 cells for second round.

In a fourth procedure, phage Fabs amplified from the Dyax libraries were panned on soluble biotin-PD-1-Fc and captured by magnetic strep-beads.

Five unique clones (R3A1, R3A2, R4B3, R3B7, and R3D6) were converted to IgG for the further characterization. The amino acid sequences of four of these variants (R3A1, R3A2, R4B3, and R3D6) are set forth in the sequence listing as indicated in rows 1-4 of Table 1.

TABLE 1
Antibody Amino Acid Sequences by SEQ ID NO.

| | V_{H} CDRs | | | | | | | | | |
|---|---|---|---|---|---|---|---|---|---|---|
| | H1 | H1 | H2 | H2 | | | V_{L} CDRs | | | |
| Mab | (K) | (C) | (K) | (C) | H3 | V_{H} | L1 | L2 | L3 | V_{L} |
| R3A1 | 1 | 2 | 3 | 4 | 5 | 6 | 7 | 8 | 9 | 10 |
| R3A2 | 11 | 12 | 13 | 14 | 15 | 16 | 17 | 18 | 19 | 20 |
| R4B3 | 21 | 22 | 23 | 24 | 25 | 26 | 27 | 28 | 29 | 30 |
| R3D6 | 31 | 32 | 33 | 34 | 35 | 36 | 37 | 38 | 39 | 40 |
| A1_101 | 1 | 2 | 3 | 4 | 5 | 6 | 41 | 42 | 43 | 44 |
| A1_102 | 1 | 2 | 3 | 4 | 5 | 6 | 45 | 46 | 47 | 48 |
| A1_103 | 1 | 2 | 3 | 4 | 5 | 6 | 49 | 50 | 51 | 52 |
| A1_104 | 1 | 2 | 3 | 4 | 5 | 6 | 53 | 54 | 55 | 56 |
| A1_105 | 1 | 2 | 3 | 4 | 5 | 6 | 57 | 58 | 59 | 60 |
| A1_106 | 1 | 2 | 3 | 4 | 5 | 6 | 61 | 62 | 63 | 64 |
| A1_107 | 1 | 2 | 3 | 4 | 5 | 6 | 65 | 66 | 67 | 68 |
| A1_108 | 1 | 2 | 3 | 4 | 5 | 6 | 69 | 70 | 71 | 72 |
| A1_109 | 1 | 2 | 3 | 4 | 5 | 6 | 73 | 74 | 75 | 76 |
| A2_101 | 11 | 12 | 13 | 14 | 15 | 16 | 77 | 78 | 79 | 80 |
| A2_102 | 11 | 12 | 13 | 14 | 15 | 16 | 81 | 82 | 83 | 84 |
| A2_103 | 11 | 12 | 13 | 14 | 15 | 16 | 85 | 86 | 87 | 88 |
| A2_104 | 11 | 12 | 13 | 14 | 15 | 16 | 89 | 90 | 91 | 92 |
| A2_105 | 11 | 12 | 13 | 14 | 15 | 16 | 93 | 94 | 95 | 96 |
| A2_106 | 11 | 12 | 13 | 14 | 15 | 16 | 97 | 98 | 99 | 100 |
| A2_107 | 11 | 12 | 13 | 14 | 15 | 16 | 101 | 102 | 103 | 104 |
| A2_108 | 11 | 12 | 13 | 14 | 15 | 16 | 105 | 106 | 107 | 108 |
| A2_109 | 11 | 12 | 13 | 14 | 15 | 16 | 109 | 110 | 111 | 112 |
| A2_110 | 11 | 12 | 13 | 14 | 15 | 16 | 113 | 114 | 115 | 116 |
| A2_111 | 11 | 12 | 13 | 14 | 15 | 16 | 117 | 118 | 119 | 120 |
| A2_112 | 11 | 12 | 13 | 14 | 15 | 16 | 121 | 122 | 123 | 124 |
| A2_113 | 11 | 12 | 13 | 14 | 15 | 16 | 125 | 126 | 127 | 128 |
| A2_114 | 11 | 12 | 13 | 14 | 15 | 16 | 129 | 130 | 131 | 132 |
| A2_115 | 11 | 12 | 13 | 14 | 15 | 16 | 133 | 134 | 135 | 136 |
| A2_116 | 11 | 12 | 13 | 14 | 15 | 16 | 137 | 138 | 139 | 140 |
| A2_117 | 11 | 12 | 13 | 14 | 15 | 16 | 141 | 142 | 143 | 144 |
| A2_118 | 11 | 12 | 13 | 14 | 15 | 16 | 145 | 146 | 147 | 148 |
| A2_119 | 11 | 12 | 13 | 14 | 15 | 16 | 149 | 150 | 151 | 152 |
| A2_120 | 11 | 12 | 13 | 14 | 15 | 16 | 153 | 154 | 155 | 156 |
| A2_121 | 11 | 12 | 13 | 14 | 15 | 16 | 157 | 158 | 159 | 160 |
| A2_122 | 11 | 12 | 13 | 14 | 15 | 16 | 161 | 162 | 163 | 164 |
| A2_123 | 11 | 12 | 13 | 14 | 15 | 16 | 165 | 166 | 167 | 168 |
| A2_124 | 11 | 12 | 13 | 14 | 15 | 16 | 169 | 170 | 171 | 172 |
| B3_101 | 21 | 22 | 23 | 24 | 25 | 26 | 173 | 174 | 175 | 176 |
| B3_102 | 21 | 22 | 23 | 24 | 25 | 26 | 177 | 178 | 179 | 180 |
| B3_103 | 21 | 22 | 23 | 24 | 25 | 26 | 181 | 182 | 183 | 184 |
| B3_104 | 21 | 22 | 23 | 24 | 25 | 26 | 185 | 186 | 187 | 188 |
| B3_105 | 21 | 22 | 23 | 24 | 25 | 26 | 189 | 190 | 191 | 192 |
| B3_106 | 21 | 22 | 23 | 24 | 25 | 26 | 193 | 194 | 195 | 196 |
| B3_107 | 21 | 22 | 23 | 24 | 25 | 26 | 197 | 198 | 199 | 200 |
| B3_108 | 21 | 22 | 23 | 24 | 25 | 26 | 201 | 202 | 203 | 204 |
| B3_109 | 21 | 22 | 23 | 24 | 25 | 26 | 205 | 206 | 207 | 208 |
| B3_110 | 21 | 22 | 23 | 24 | 25 | 26 | 209 | 210 | 211 | 212 |
| D6_101 | 31 | 32 | 33 | 34 | 35 | 36 | 213 | 214 | 215 | 216 |
| D6_102 | 31 | 32 | 33 | 34 | 35 | 36 | 217 | 218 | 219 | 220 |
| D6_103 | 31 | 32 | 33 | 34 | 35 | 36 | 221 | 222 | 223 | 224 |
| D6_104 | 31 | 32 | 33 | 34 | 35 | 36 | 225 | 226 | 227 | 228 |
| D6_105 | 31 | 32 | 33 | 34 | 35 | 36 | 229 | 230 | 231 | 232 |
| D6_106 | 31 | 32 | 33 | 34 | 35 | 36 | 233 | 234 | 235 | 236 |
| D6_107 | 31 | 32 | 33 | 34 | 35 | 36 | 237 | 238 | 239 | 240 |
| D6_108 | 31 | 32 | 33 | 34 | 35 | 36 | 241 | 242 | 243 | 244 |
| D6_109 | 31 | 32 | 33 | 34 | 35 | 36 | 245 | 246 | 247 | 248 |

### Antibody Characterization

The binding activity of the five antibodies was examined using dose-response ELISA. The serially diluted antibodies were added to immobilized hPD-1-Fc and detected by HRP conjugated anti-hIgG Fab specific antibody. The OD450 reading was plotted vs. log antibody concentration using GraphPad Prism6. EC50 was calculated by using “dose-response” (stimulation) variable slope (four parameters)” program. The antibodies were also added to immobilized mPD-1-Fc and other Fc-fusions to determine specificity for hPD-1-Fc and to immobilized anti-human IgG fc specific antibody (anti-hFc Ab) to check the accuracy of the concentration measurement.

The interactions of ligands and receptor were examined in the presence of serially diluted antibodies by dose response blocking ELISA. Mixtures of the serially diluted antibodies, with fixed amount of receptor hPD-1 (0.25 μg/ml final), were added to ligand hPD-L1 coated plates. The amount of hPD-1 bound on the ligands was quantified by incubation with Biotin-anti-human PD-1 polyclone antibody (goat), then with Strep-HRP. The OD450 reading was plotted vs. log antibody concentration by using GraphPad Prism6. IC50 was calculated by using “dose response (inhibition) variable slope (four parameters)” program.

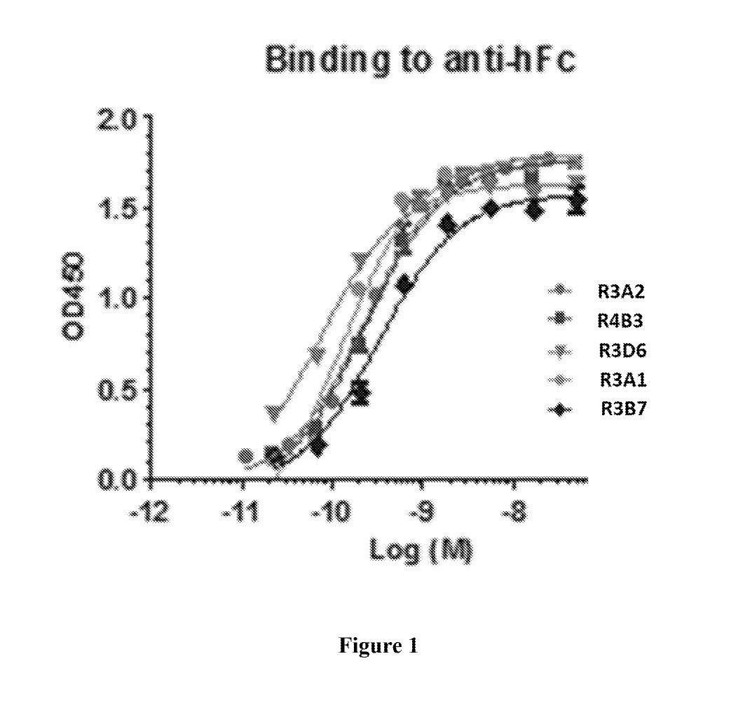

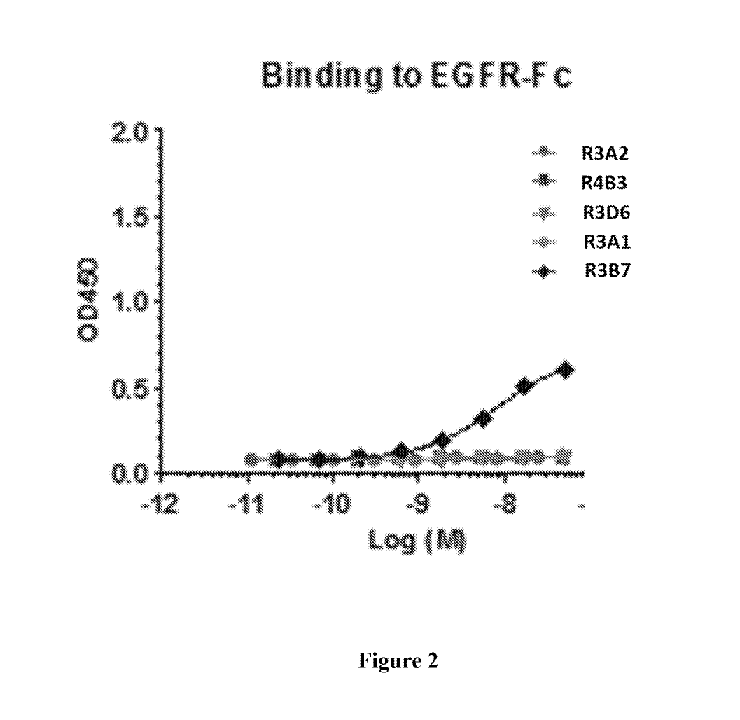

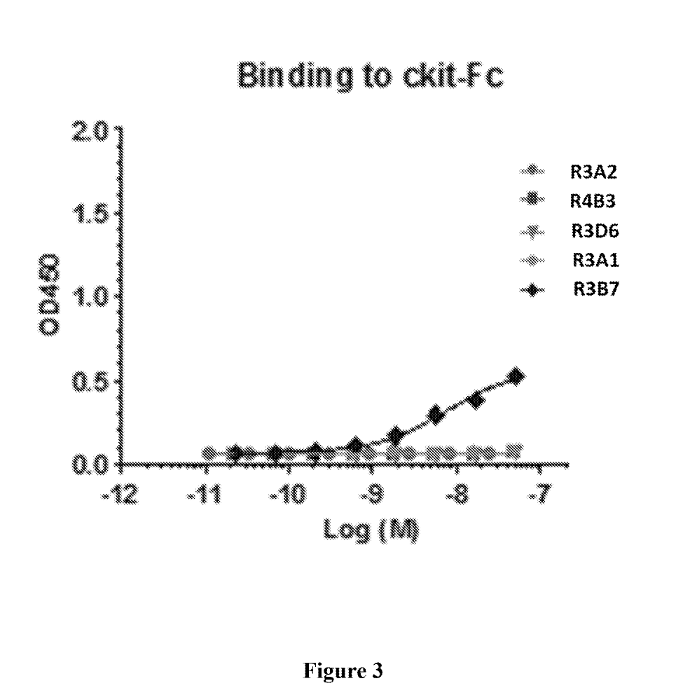

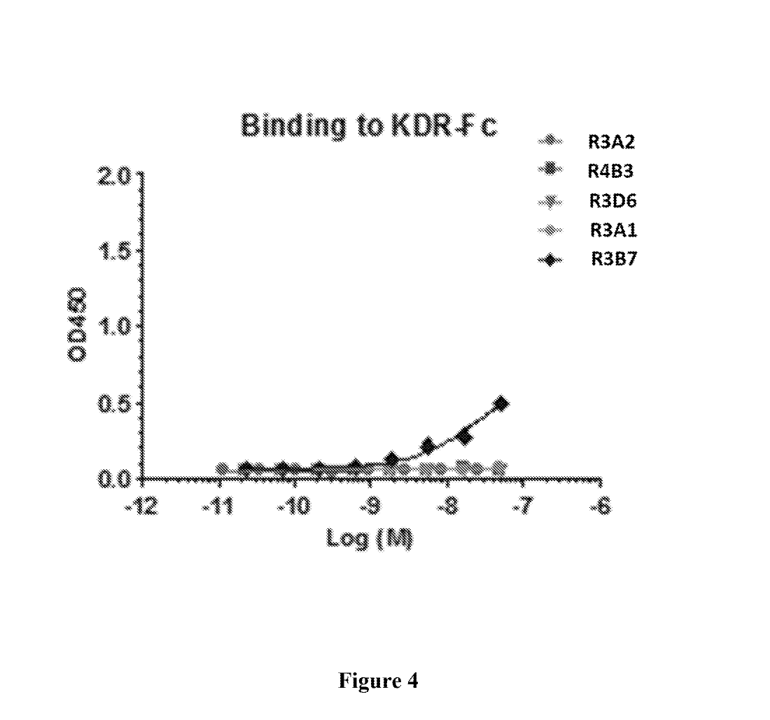

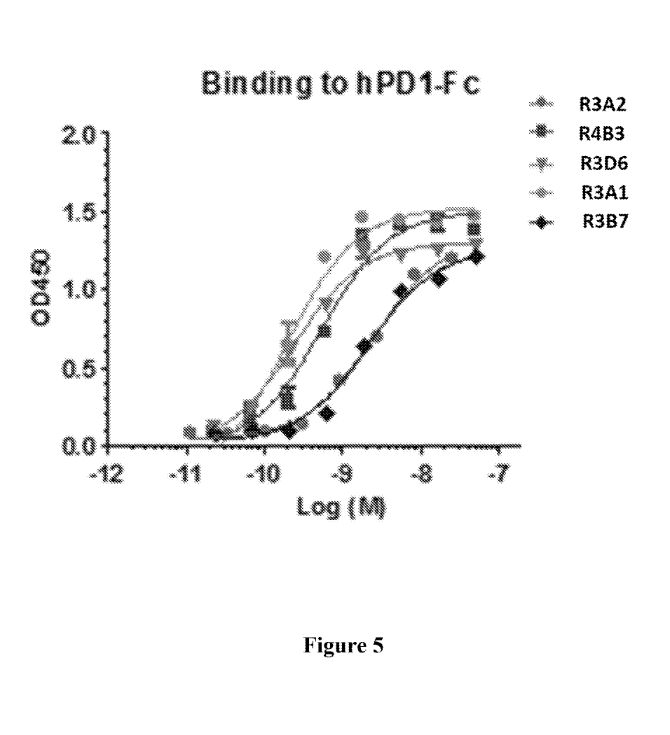

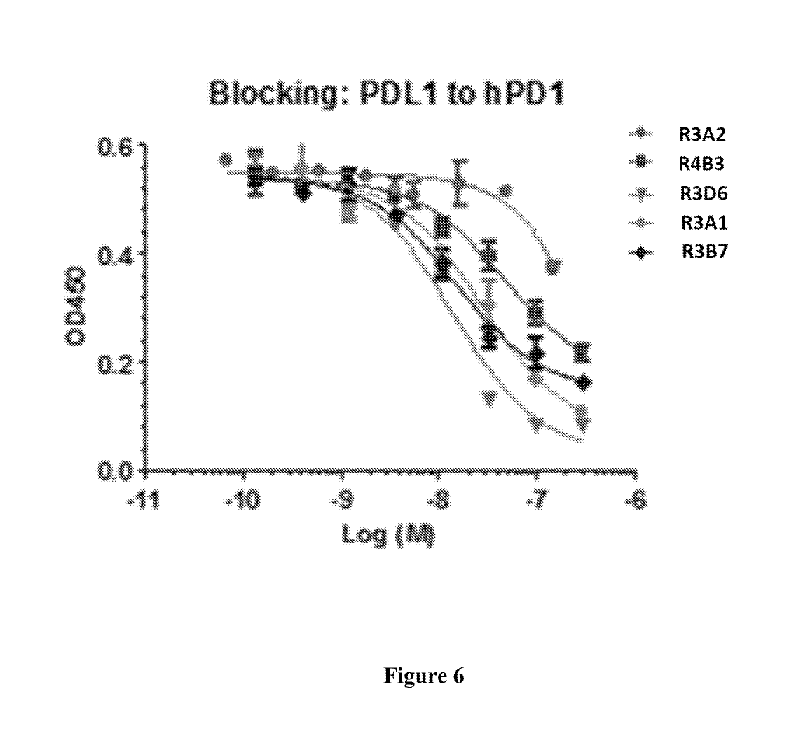

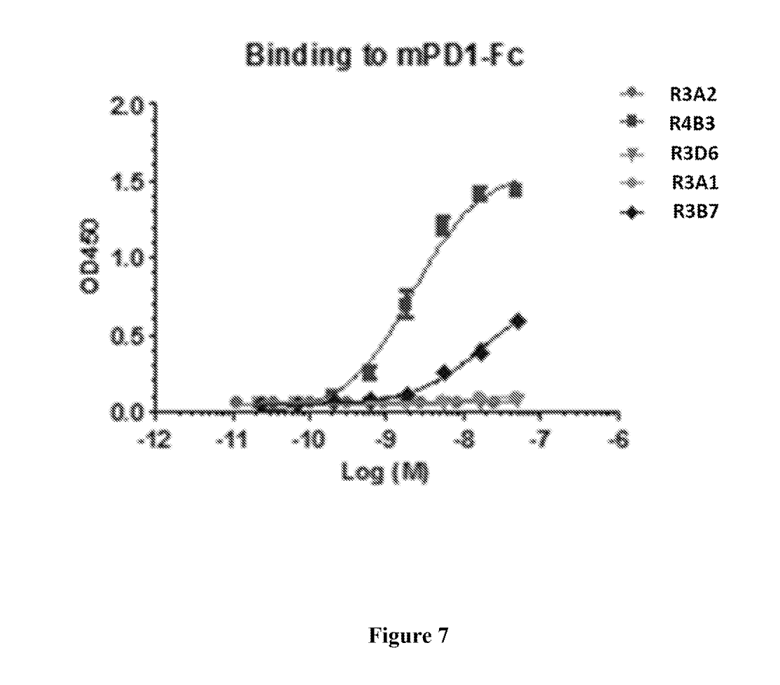

Results are shown in FIGS. 1-7. All five antibodies can bind to soluble human PD-1-Fc (FIG. 5) and block ligand PD-L1 binding to PD-1 (FIG. 6). R4B3 can also bind to murine PD-1 (FIG. 7), and R3B7 has low binding affinity to other Fc-fusions (FIGS. 2-4). R3A1, R3A2, R4B3, and R3D6 were carried forward for affinity maturation through light chain shuffling.

### Affinity Maturation

Light chain shuffling was used to increase the affinity of the four lead antibodies. More particularly, the heavy chains of R3A1, R3A2, R4B3, and R3D6 were paired with κλ light chain pool to build light chain shuffling libraries. The libraries were panned for higher affinity antibodies. The ELISA positive clones were sequenced. The unique clones were compared by competition ELISA. The amino acid sequences of these variants are set forth the sequence listing as indicated in rows 5-56 of Table 1.

### Lead Characterization

ELISA

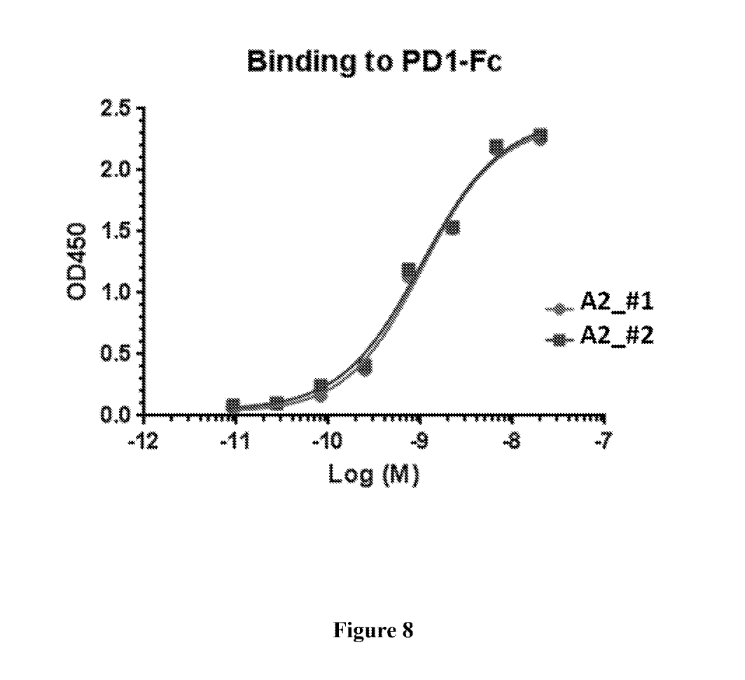

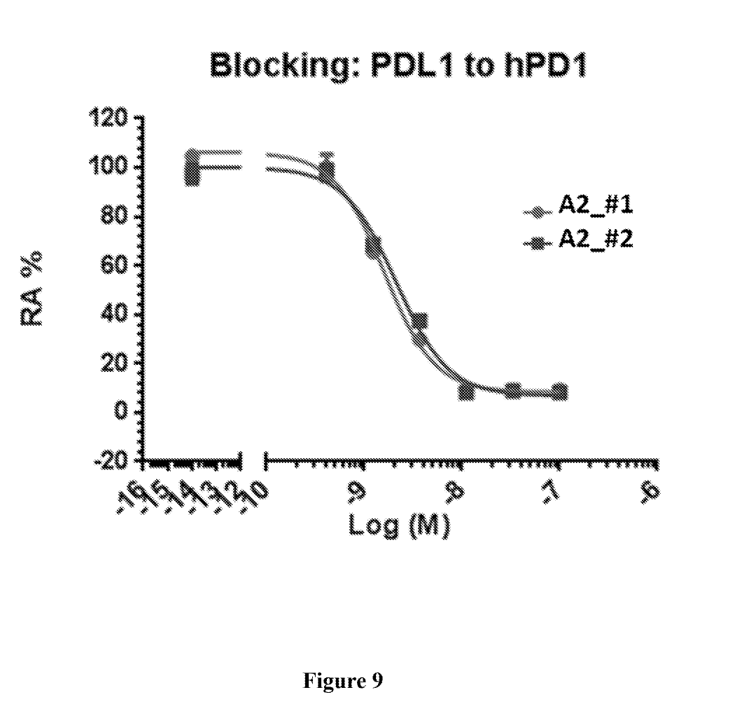

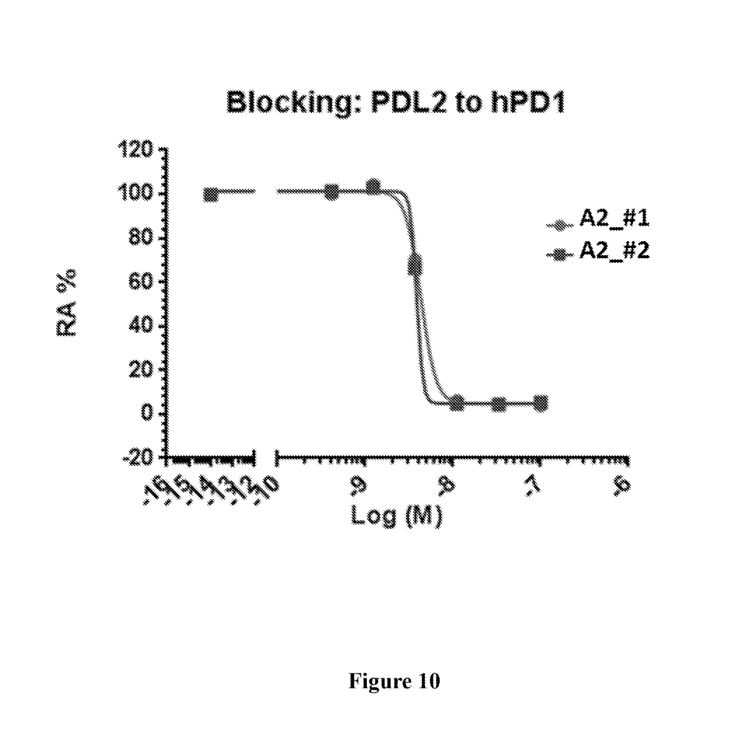

Two lead candidates from the R3A2 shuffling libraries were characterized using solid-phase ELISA as described above. Results are shown in FIGS. 8-10. A2_#1 and A2_#2 can bind to hPD-1 strongly (FIG. 8) and block ligand PD-L1 (FIG. 9) and PD-L2 (FIG. 10) binding to its receptor PD-1. EC50 and IC50 values are shown in Table 2.

TABLE 2
EC₅₀ and IC₅₀ for antibodies of FIG. 2

| | A2_#1 | A2_#2 |
|---|---|---|
| EC50 to hPD-1-Fc (nM) | 1.54 | 3.34 |
| IC50, hPD-L1 to hPD-1 (nM) | 1.63 | 4.29 |
| IC50, hPD-L2 to hPD-1 (nM) | 2.13 | 3.87 |

Biacore

The binding kinetics of the two lead candidates were evaluated using surface plasmon resonance (SPR) on a Biacore T-200 instrument (GE Healthcare). A CMS chip was equilibrated in running buffer HBSEP (running buffer) at 10 μl/ml. Two flow cells of a CMS chip were activated with 1:1 NHS/EDC injection for five minutes. The second flow cell was immobilized with 5 μg/ml of hPD-L1fc diluted in 10 mM sodium acetate buffer pH 5 to reach 30-50RU of immobilized protein. The surfaces were subsequently blocked with a 5 minute injection of ethanolamine followed by a 5 minute injection of NSB reducer.

The sample runs were performed in HBSEP running buffer at 30 μl/min. Samples were serially diluted in running buffer at concentrations ranging from 100-1.37 nM. Samples were injected over the two flow cells for an association time of three minutes and a dissociation time of 10 minutes. Regeneration was performed after each binding cycle with a 30 second injection of 20 mM HCL. Sensorgrams were obtained for each concentration and the derived curves were fitted to a 1:1 Langmuir binding model with a blank flow cell subtraction using the Biaevaluation software. Results are shown in Table 3.

TABLE 3
Kinetic Analysis for antibodies of FIG. 2

| | | ka | kd | kD |
|---|---|---|---|---|
| A2_#1 | 1 | 7.82E+04 | 9.04E−08 | 1.16E−12 |
| | 2 | 7.25E+04 | 1.28E−05 | 1.77E−10 |
| | Average | 7.54E+04 | 6.45E−06 | 8.91E−11 |
| A2_#2 | 1 | 1.36E+05 | 2.14E−05 | 1.57E−10 |
| | 2 | 1.22E+05 | 2.10E−05 | 1.72E−10 |
| | Average | 1.29E+05 | 2.12E−05 | 1.65E−10 |

Flow Cytometry for Binding Activity of Anti-PD-1 Antibodies to Cell Surface Expressed PD-1, PD-L1 Receptor

The binding activity of the lead candidate antibodies to surface expressed PD-1 or PD-L1 in HEK293 transfectant cells and CD4 and CD8 T cells was evaluated by flow cytometry using Guava EasyCyte™ HT Sampling Flow Cytometer (EMD Millipore) per manufacture instruction. Purified human CD4 and CD8 T cells were activated by anti-CD3 antibody coated beads at 1:5 ratio of bead to T cells for 4 days prior to staining with anti-PD-1 antibodies. R-phycoerythrin-conjugated goat anti-human IgG (Cat#109-116-098, Jackson ImmunoResearch) was used as secondary antibody to detect the binding of primary antibodies.

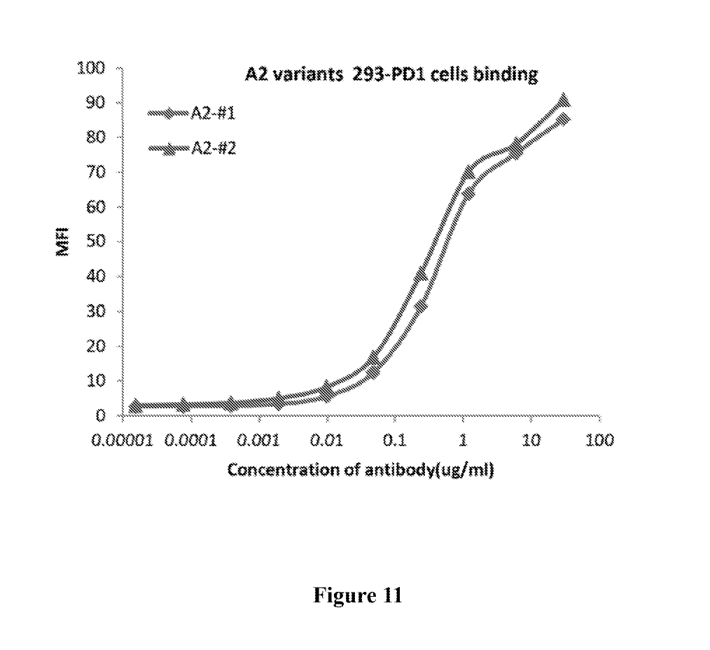

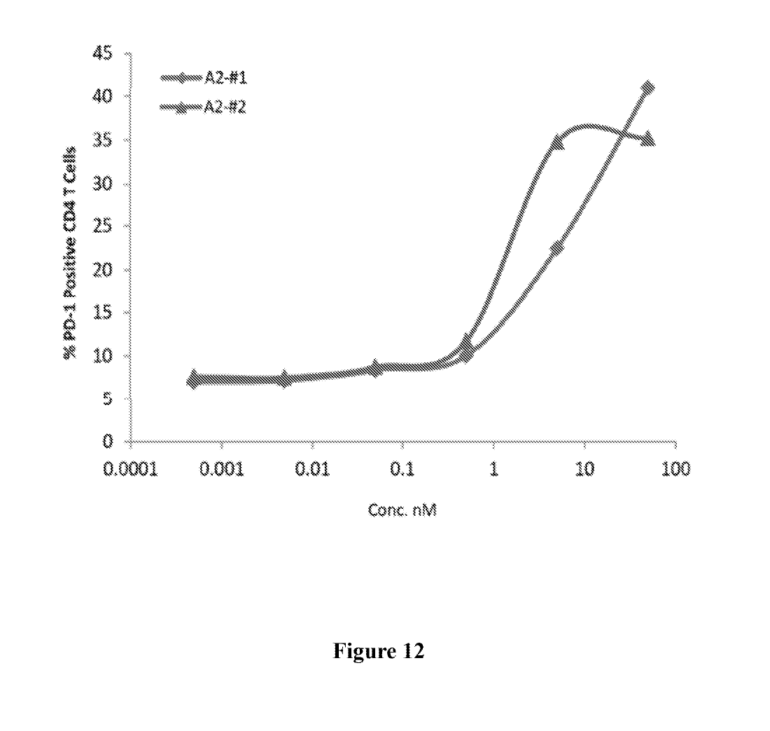

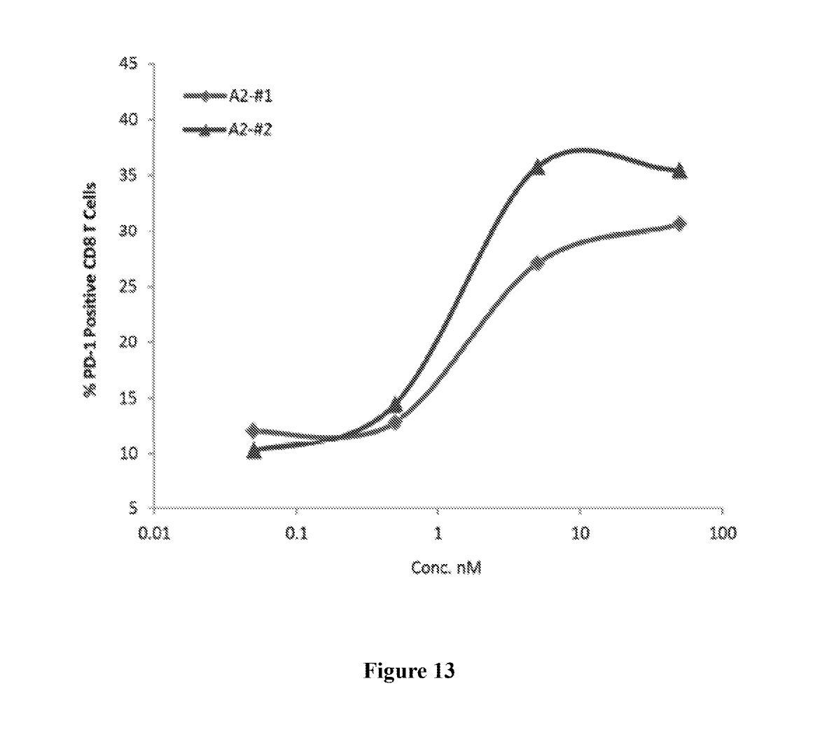

Results for HEK293 cells are shown in FIG. 11, for CD4 T cells in FIG. 12, and CD8 T cells in FIG. 13.

SEB Activated PBMC Assay for Cytokine Production and T Cell Proliferation

Anti-PD-1 antibodies A2_#1 and A2_#2 were verified to have comparable activity to increase Th1 cytokine secretion. PBMCs were isolated from LeukoPak (an enriched leukapheresis containing highly concentrated blood cells including monocytes, lymphocytes, platelets, plasma, as well as red cells using Histopaque-1077 (Sigma) per manufacture instruction. PBMCs were cultured at 2×10⁴ per well in 96 well plate containing IMDM (Iscove's Modified Dulbecco's Medium, supplemented 2 mM Glutamine 25 mM HEPES, 3.024 g/L Sodium Bicarbonate, Technologies cat#31980-097) and 10% Fetal Bovine Serum (FBS, cat# SH30396.03, HyClone) and activated by 0.1 ug/mL SEB (Staphylococcal enterotoxin B, Sigma) for 5 to 7 days. The effect of the lead candidate anti-PD-1 antibodies on cytokine production in SEB activated PBMC was assessed by the measurement of cytokine released in the culture. At day 7, supernatants were collected for the measurements of IL-2 and IFNγ using Duoset ELISA Kit (R&D Systems) per manufacture instruction.

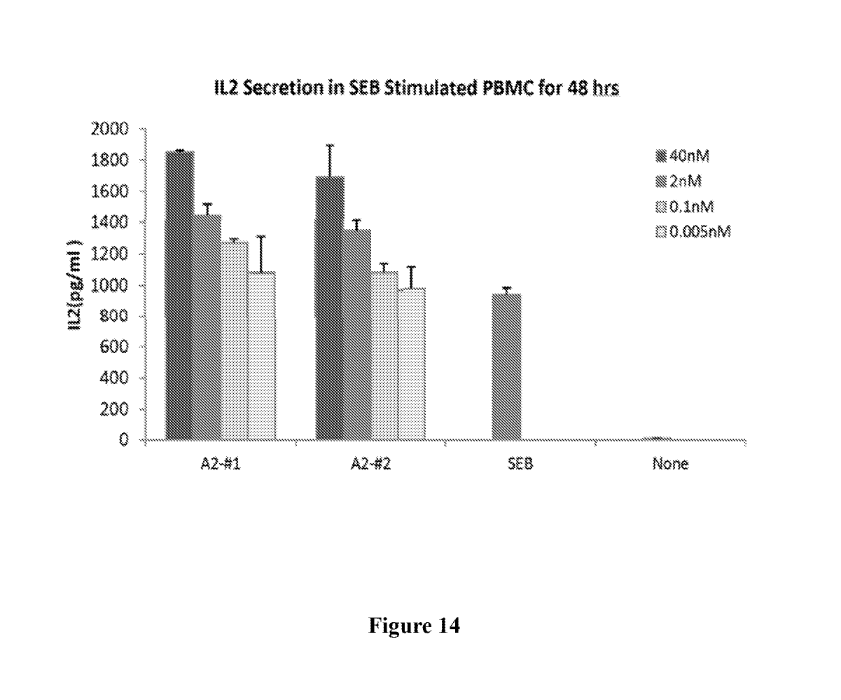

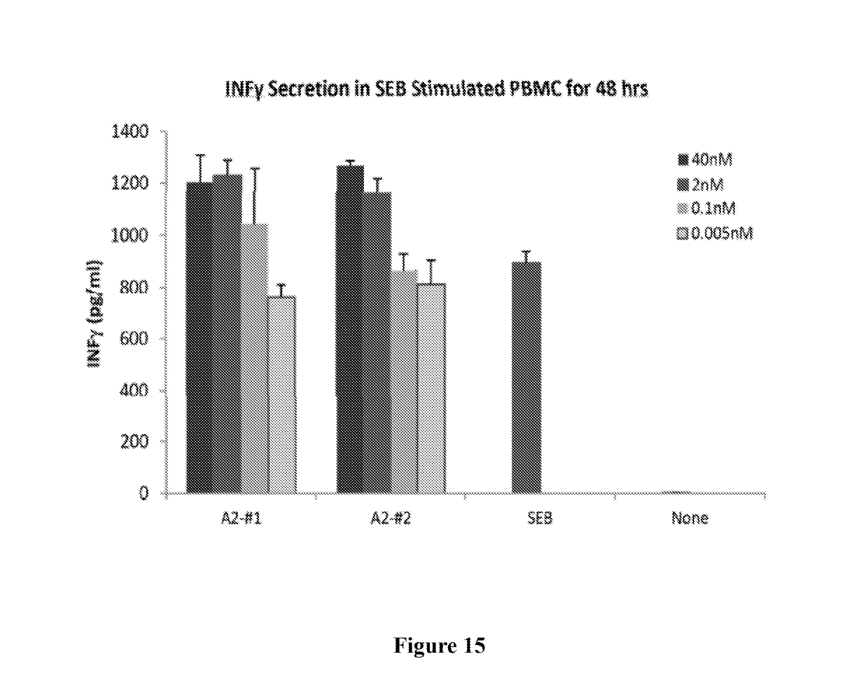

Results for IL-2 secretion are shown in FIG. 14, and results for IFNγ are shown in FIG. 15. Signficant increases in the levels of IFNγ were observed in cultures with the lead candidate anti-PD-1 antibodies A2_#1 and A2_#2.

Mixed-Lymphocyte Reaction Assay for Cytokine Production and T Cell Proliferation

Anti-PD-1 antibodies A2_#1 and A2_#2 were verified to have comparable activity to increase the proliferation of CD4 T cells. Human CD4 T cells isolated from whole blood were stimulated with anti-CD3 antibody and PD-L1-Fc coated beads in the presence of anti-PD-1 antibodies for 4 days. Proliferation was measured by proliferative marker Ki67 staining.

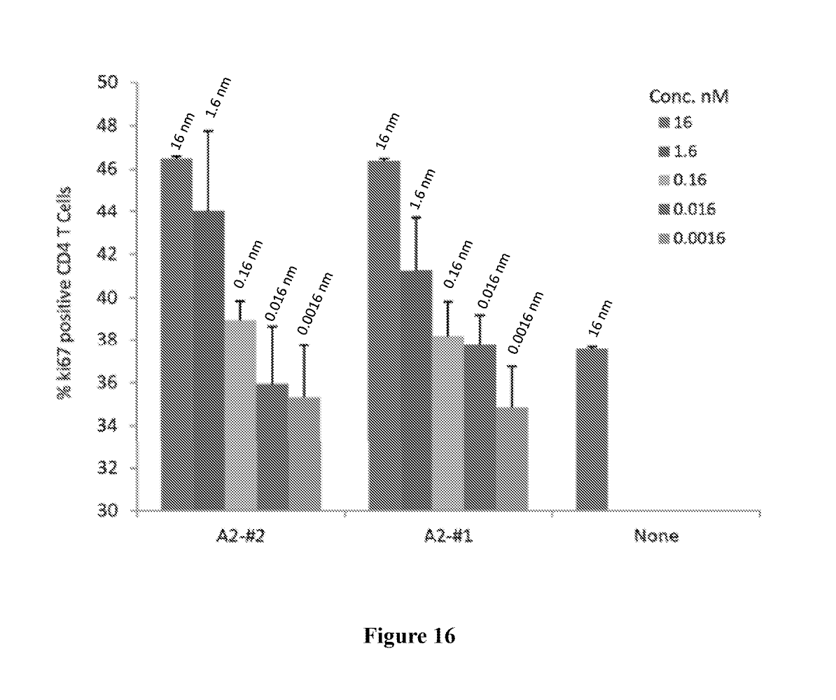

Results are shown in FIG. 16. Significant increases in the proliferation of CD4 T cells were observed in cultures with anti-PD-1 antibodies A2 #1 and A2 #2.

Anti-PD-1 antibodies A2_#1 and A2_#2 were verified to have comparable activity to increase Th1 cytokine secretion in Mixed Lymphocyte reactions. Immature monocyte-derived dendritic cells (mo-DC) were generated by culturing CD14 positive cells in IMDM supplemented with 10% FBS with 150 ng/ml GM-CSF and 50 ng/mL IL-4 for 6 to 7 days. CD4 positive cells were negatively isolated from whole blood using RosetteSep human CD4 enrichment kit (StemCell Technologies). Mo-DC and CD4 positive cells were then co-cultured at a ratio 1:10 of mo-DC to CD4 cells, respectively. To assess blocking function of anti-PD-1 antibodies, increasing amount of anti-PD-1 antibodies was added in the beginning of co-culture. At day 7, the supernatants were collected for measurements of secreted IFNγ by ELISA.

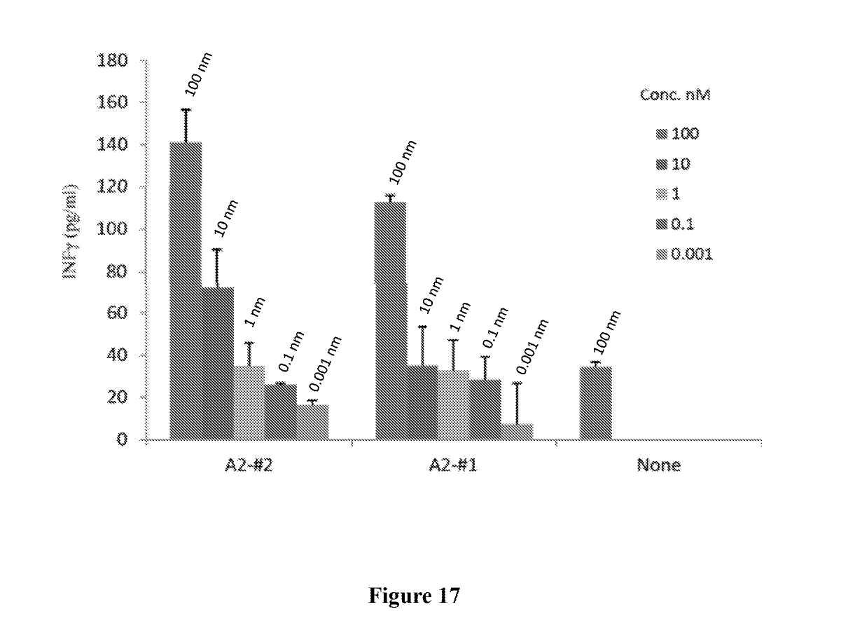

Results are shown in FIG. 17. Significant increases in the levels of IFNγ were observed in cultures with the variants of anti-PD-1 antibodies A2 #1 and A2 #2.

What is claimed is:

1. An antibody or fragment thereof that binds to PD-1, which comprises a heavy chain variable domain with a CDR-1H, a CDR-2H, and a CDR-3H as set forth in Table 1.

2. An antibody or fragment thereof that binds to PD-1, which comprises a light chain variable domain with a CDR-1L, a CDR-2L, and a CDR-3L as set forth in Table 1.

3. The antibody or fragment of claim 1, which comprises a light chain variable domain with a CDR-1L, a CDR-2L, and a CDR-3L as set forth in Table 1.

4. An antibody or fragment thereof that binds to PD-1, which comprises a heavy chain variable domain sequence set forth in Table 1, or a heavy chain variable domain sequence set forth in Table 1 with conservative substitutions such that it is at least 80% identical to the heavy chain variable domain sequence set forth in Table 1.

5. The antibody or fragment thereof of claim 4, which comprises a heavy chain variable domain sequence set forth in Table 1 with conservative substitutions such that it is at least 85% identical to the heavy chain variable domain sequence set forth in Table 1.

6. The antibody or fragment thereof of claim 4, which comprises a heavy chain variable domain sequence set forth in Table 1 with conservative substitutions such that it is at least 90% identical to the heavy chain variable domain sequence set forth in Table 1.

7. The antibody or fragment thereof of claim 4, which comprises a heavy chain variable domain sequence set forth in Table 1 with conservative substitutions such that it is at least 95% identical to the heavy chain variable domain sequence set forth in Table 1.

8. An antibody or fragment thereof that binds to PD-1, which comprises a light chain variable domain sequence set forth in Table 1, or a light chain variable domain sequence set forth in Table 1 with conservative substitutions such that is at least 80% identical to the light chain variable domain sequence set forth in Table 1.

9. The antibody or fragment thereof of claim 8, which comprises a light chain variable domain sequence set forth in Table 1 with conservative substitutions such that it is at least 85% identical to the light chain variable domain sequence set forth in Table 1.

10. The antibody or fragment thereof of claim 8, which comprises a light chain variable domain sequence set forth in Table 1 with conservative substitutions such that it is at least 90% identical to the light chain variable domain sequence set forth in Table 1.

11. The antibody or fragment thereof of claim 8, which comprises a light chain variable domain sequence set forth in Table 1 with conservative substitutions such that it is at least 95% identical to the light chain variable domain sequence set forth in Table 1.

12. The antibody or fragment of claim 4, which comprises a light chain variable domain sequence set forth in Table 1, or a light chain variable domain sequence set forth in Table 1 with conservative substitutions such that is at least 80% identical to the light chain variable domain sequence set forth in Table 1.

13. The antibody or fragment thereof of claim 12, which comprises a light chain variable domain sequence set forth in Table 1 with conservative substitutions such that it is at least 85% identical to the light chain variable domain sequence set forth in Table 1.

14. The antibody or fragment thereof of claim 12, which comprises a light chain variable domain sequence set forth in Table 1 with conservative substitutions such that it is at least 90% identical to the light chain variable domain sequence set forth in Table 1.

15. The antibody or fragment thereof of claim 12, which comprises a light chain variable domain sequence set forth in Table 1 with conservative substitutions such that it is at least 95% identical to the light chain variable domain sequence set forth in Table 1.

16. A conjugate of the antibody or fragment of any one of claims 1 to 15, further comprising an imaging agent, a therapeutic agent, or a cytotoxic agent.

17. An isolated nucleic acid sequence that encodes an antibody variable domain or fragment of any one of claims 1 to 15.

18. A nucleic acid vector comprising the nucleic acid of claim 17.

19. A prokaryotic or eukaryotic host cell comprising the nucleic acid of claim 17.

20. A composition comprising an antibody or fragment of any one of claims 1 to 15 and a pharmaceutically acceptable carrier.

21. A method of inhibiting the interaction of PD-1 with PD-L1 in a subject, which comprises administering an effective amount of an antibody or fragment of any one of claims 1 to 15.

22. A method of inhibiting immunosuppression mediated by PD-1 in a subject, which comprises administering an effective amount of the antibody or fragment of any one of claims 1 to 15.

23. A method of stimulating an immune response against a cell that expresses PD-1, which comprises administering to a subject an effective amount of the antibody or fragment of any one of claims 1 to 15.

24. The method of claim 23, wherein the cell that expresses PD-1 is a tumor cell.

25. The method of claim 23, wherein the cell that expresses PD-1 is infected with a virus.
